(19) United States
(12) Patent Application Publication
(10) Pub. No.: US 2024/0400581 A1
(43) Pub. Date: Dec. 5, 2024

(54) Process for Preparing a Macrocyclic Compound Having ENT1 Inhibiting Activity

(71) Applicant: iTeos Belgium SA, Gosselies (BE)

(72) Inventors: Michael Deligny, Rhode Saint Genese (BE); Eric Talbot, Hoddesdon (GB); Mustafa Moroglu, Hoddesdon (GB); Didier Roche, Lyon (FR); Hugues Lemoine, Lyon (FR); Xin Huang, Kowloon (HK); Cuicui Yuan, Kowloon (HK); Deju Shang, Kowloon (HK)

(73) Assignee: iTeos Belgium SA, Gosselies (BE)

(21) Appl. No.: 18/624,336

(22) Filed: Apr. 2, 2024

Related U.S. Application Data
(63) Continuation of application No. PCT/CN2022/123711, filed on Oct. 5, 2022.

(30) Foreign Application Priority Data
Oct. 6, 2021 (WO) PCT/CN2021/122508
Oct. 6, 2021 (WO) PCT/CN2021/122511
Oct. 6, 2021 (WO) PCT/CN2021/122512

(51) Int. Cl. C07D 498/08

(52) U.S. Cl. CPC C07D 498/08

(57) ABSTRACT

The present disclose includes, among other things, methods of preparing compound (R)-11.

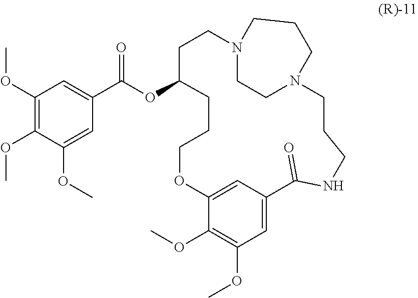 [SMILES: COc1cc(C(=O)O[C@@H]2CCCOc3cc(cc(OC)c3OC)C(=O)NCCCN3CCCN(CC2)CC3)cc(OC)c1OC]

or a pharmaceutically acceptable salt thereof. Compound (R)-11 is useful to treat or lessen the severity of cancer.

This application is a continuation of International Application No. PCT/CN2022/123711, filed Oct. 5, 2022, which claims the benefit of International Application No. PCT/CN2021/122508, filed Oct. 6, 2021, International Application No. PCT/CN2021/122511, filed Oct. 6, 2021, and International Application No. PCT/CN2021/122512, filed Oct. 6, 2021, all of which are herein incorporated by reference in their entirety.

### FIELD

The present disclosure relates to synthesis of macrocyclic diamines. More particularly, the present disclosure relates to the manufacture of inhibitors of ENT family transporter, especially of ENT1, that are useful as therapeutic compounds, especially in the treatment of cancers.

### BACKGROUND

The equilibrative nucleoside transporter (ENT) family, also known as SLC29, is a group of plasmalemmal transport proteins which transport nucleoside substrates into cells. There are four known ENTs, designated ENT1, ENT2, ENT3, and ENT4.

One of the endogenous substrates for ENTs is adenosine, a potent physiological and pharmacological regulator of numerous functions. Cellular signaling by adenosine occurs through four known G-protein-coupled adenosine receptors A1, A2A, A2B, and A3. By influencing the concentration of adenosine available to these receptors, ENTs fulfil important regulatory roles in different physiological processes, such as modulation of coronary blood flow, inflammation, and neurotransmission (Griffith D A and Jarvis S M, Biochim Biophys Acta, 1996, 1286, 153-181; Shryock J C and Belardinelli L, Am J Cardiol, 1997, 79 (12A), 2-10; Anderson C M et al., J Neurochem, 1999, 73, 867-873).

Adenosine is also a potent immunosuppressive metabolite that is often found elevated in the extracellular tumor microenvironment (TME) (Blay J et al., Cancer Res, 1997, 57, 2602-2605). Extracellular adenosine is generated mainly by the conversion of ATP by the ectonucleotidases CD39 and CD73 (Stagg J and Smyth M J, Oncogene, 2010, 2, 5346-5358). Adenosine activates four G-protein-coupled receptor subtypes (A1, A2A, A2B, and A3). In particular, activation of the A2A receptor is believed to be the main driver of innate and adaptive immune cell suppression leading to suppression of antitumor immune responses (Ohta and Sitkovsky, Nature, 2001, 414, 916-920) (Stagg and Smyth, Oncogene, 2010, 2, 5346-5358) (Antonioli L et al., Nature Reviews Cancer, 2013, 13, 842-857) (Cekic C and Linden J, Nature Reviews, Immunology, 2016, 16, 177-192) (Allard B et al., Curr Op Pharmacol, 2016, 29, 7-16) (Vijayan D et al., Nature Reviews Cancer, 2017, 17, 709-724).

The Applicant previously evidenced in PCT/EP2019/076244 that adenosine as well as ATP profoundly suppress T cell proliferation and cytokine secretion (IL-2), and strongly reduce T cell viability. Adenosine- and ATP-mediated suppression of T cell viability and proliferation were successfully restored by using ENTs inhibitors. Moreover, the use of an ENT inhibitor in combination with an adenosine receptor antagonist enabled to restore not only adenosine- and ATP-mediated suppression of T cell viability and proliferation, but also restored T cell cytokine secretion. These results showed that ENTs inhibitors either alone or in combination with an adenosine receptor antagonist may be useful for the treatment of cancers.

A variety of drugs such as dilazep, dipyridamole, and draflazine interact with ENTs and alter adenosine levels, and were developed for their cardioprotective or vasodilatory effects.

Currently, two non-selective ENT1 inhibitors (dilazep and dipyridamole) are on the market (Vlachodimou et al., Bio-Chemical Pharmacology, 2020, 172, 113747). However, their binding kinetics are unknown; moreover, there is still a need for more potent ENTs inhibitors, and especially ENT1 inhibitors to be used for the treatment of cancers, either alone or in combination with an adenosine receptor antagonist.

Consequently, there remains a need for an efficient, cost-effective process for the production of ENT1 inhibitors in high yield. The present disclosure provides a viable method of preparing of high value key intermediates for ENT1 inhibitors.

### SUMMARY

The present disclosure includes methods of preparing compound (R)-11:

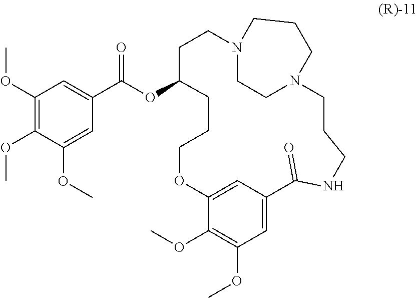 [SMILES: COc1cc(C(=O)O[C@@H]2CCCOc3cc(cc(OC)c3OC)C(=O)NCCCN3CCCN(CC2)CC3)cc(OC)c1OC]

or a pharmaceutically acceptable salt thereof.

### DETAILED DESCRIPTION

The present disclosure relates to synthesis of key intermediates which are useful in the synthesis of ENT1 inhibitors.

In general, the synthesis pathways for any individual compound of the present disclosure will depend on the specific substituents of each molecule and upon the ready availability of intermediates necessary; again such factors being appreciated by those of ordinary skill in the art. According to a further general process, compounds of the present disclosure can be converted to alternative compounds of the present disclosure, employing suitable interconversion techniques well known by a person skilled in the art. It will be understood that any step disclosed herein can be rendered enantioselective through the use of a suitable reagent. Additionally, the present disclosure contemplates the use of enantioenriched starting material(s). In some embodiments, a reaction disclosed herein that produces a chiral product could be purified using separation methods known in the art to separate one enantiomer from another.

In some embodiments, synthesis of compound (R)-11 can be accomplished in a process comprising any of steps 1-10 summarized in Scheme 1.

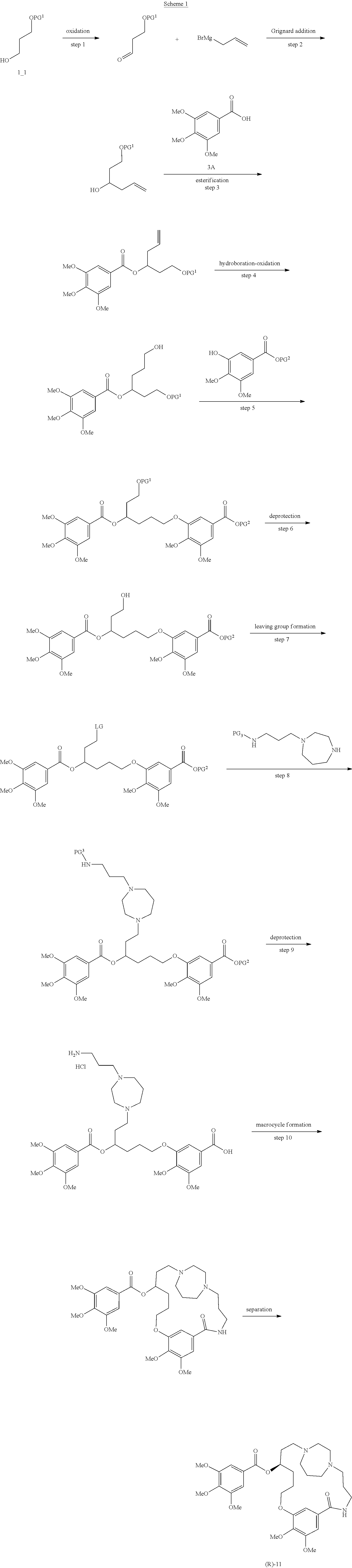

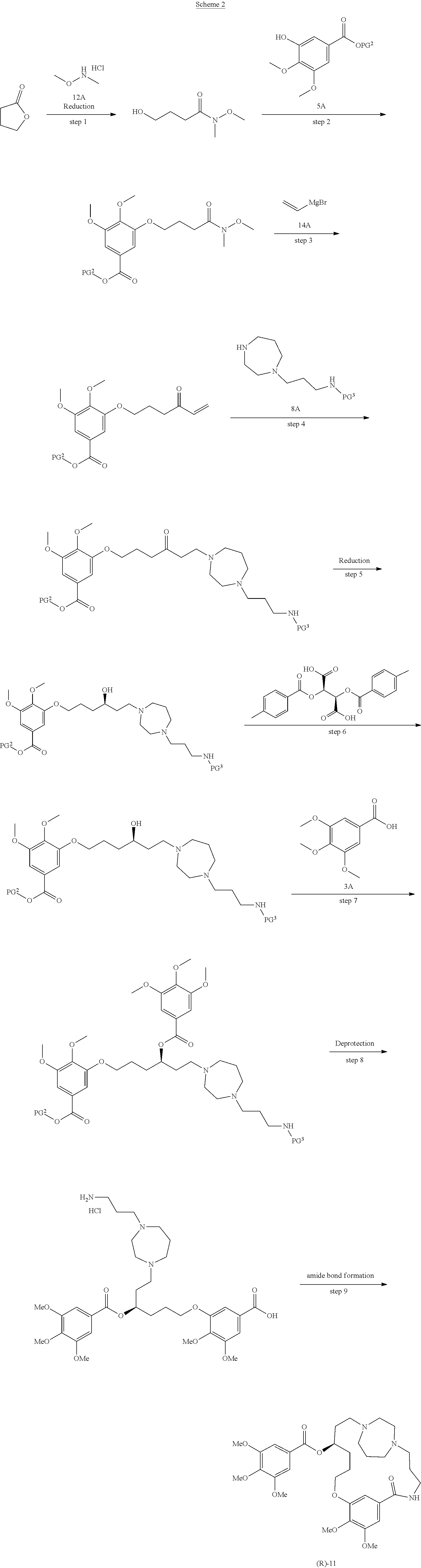

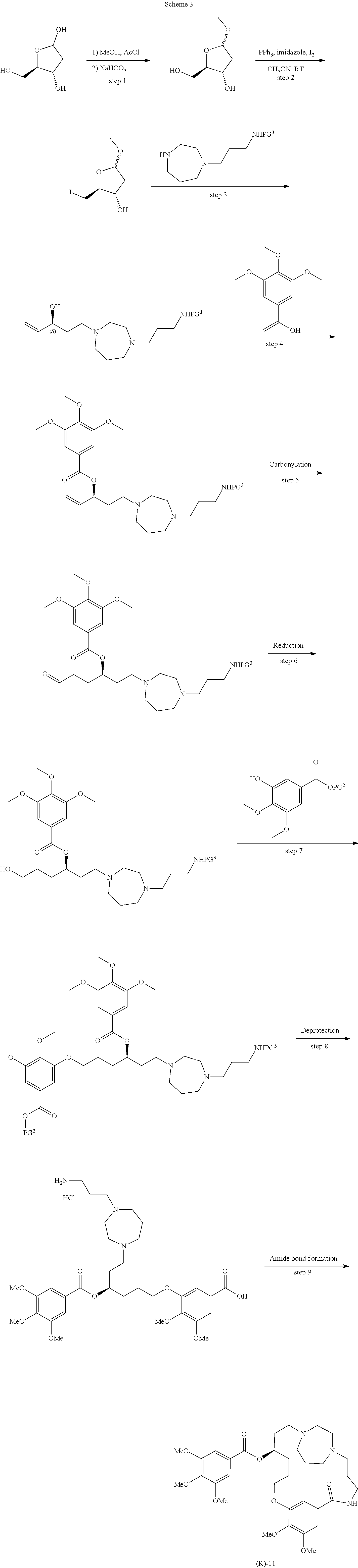

In some embodiments, PG¹ is a suitable hydroxyl protecting group. The term “hydroxyl-protecting group” is likewise known in general terms and relates to groups which are suitable for protecting a hydroxyl group against chemical reactions, but are easy to remove after the desired chemical reaction has been carried out elsewhere in the molecule. Typical of such groups are the above-mentioned unsubstituted or substituted aryl, aralkyl or acyl groups, furthermore also alkyl groups. The nature and size of the hydroxyl protecting groups are not crucial since they are removed again after the desired chemical reaction or reaction sequence; preference is given to groups having 1-20, in particular 1-10, carbon atoms. Examples of hydroxyl-protecting groups are, inter alia, benzyl, 4-methoxybenzyl, p-nitrobenzoyl, p-toluenesulfonyl, tert-butyl and acetyl, where benzyl and tert-butyl are particularly preferred.

In some embodiments, PG¹ is selected from the group consisting of Acetyl (Ac), Benzoyl (Bz), Benzyl (Bn) β-Methoxyethoxymethyl ether (MEM), Dimethoxytrityl, [bis-(4-methoxyphenyl)phenylmethyl] (DMT), Methoxymethyl ether (MOM), Methoxytrityl [(4-methoxyphenyl)diphenylmethyl] (MMT) p-Methoxybenzyl ether (PMB), p-Methoxyphenyl ether (PMP), Methylthiomethyl ether, Pivaloyl (Piv), Tetrahydropyranyl (THP), Tetrahydrofuran (THF), Trityl (triphenylmethyl, Tr), and a silyl ether. In some embodiments PG¹ is a silyl ether. In some embodiments, PG¹ is selected from the group consisting of trimethylsilyl (TMS), tert-butyldimethylsilyl (TBS), tri-iso-propylsilyloxymethyl (TOM), and triisopropylsilyl (TIPS) ethers). In some embodiments, PG¹ is tert-butyldimethylsilyl (TBS).

In some embodiments, PG² is selected from the group consisting of Acetyl (Ac), Benzoyl (Bz), Benzyl (Bn) β-Methoxyethoxymethyl ether (MEM), Dimethoxytrityl, [bis-(4-methoxyphenyl)phenylmethyl] (DMT), Methoxymethyl ether (MOM), Methoxytrityl [(4-methoxyphenyl)diphenylmethyl] (MMT) p-Methoxybenzyl ether (PMB), p-Methoxyphenyl ether (PMP), Methylthiomethyl ether, Pivaloyl (Piv), Tetrahydropyranyl (THP), Tetrahydrofuran (THF), Trityl (triphenylmethyl, Tr), and a silyl ether. In some embodiments, PG² is C₁-C₆ aliphatic. In some embodiments, PG² is t-Bu.

In some embodiments, PG³ is an amino-protecting group. The term “amino-protecting group” is known in general terms and relates to groups which are suitable for protecting (blocking) an amino group against chemical reactions, but which are easy to remove after the desired chemical reaction has been carried out elsewhere in the molecule. Typical of such groups are, in particular, unsubstituted or substituted acyl, aryl, aralkoxymethyl or aralkyl groups. Since the amino-protecting groups are removed after the desired reaction (or reaction sequence), their type and size are furthermore not crucial; however, preference is given to those having 1-20, in particular 1-8, carbon atoms. The term “acyl group” is to be understood in the broadest sense in connection with the present process. It includes acyl groups derived from aliphatic, araliphatic, aromatic or heterocyclic carboxylic acids or sulfonic acids, and, in particular, alkoxy-carbonyl, aryloxycarbonyl and especially aralkoxycarbonyl groups. Examples of such acyl groups are alkanoyl, such as acetyl, propionyl and butyryl; aralkanoyl, such as phenylacetyl; aroyl, such as benzoyl and tolyl; aryloxyalkanoyl, such as POA; alkoxycarbonyl, such as methoxy-′carbonyl, ethoxycarbonyl, 2,2,2-trichloroethoxycarbonyl, BOC (tert-butoxycarbonyl) and 2-iodoethoxycarbonyl aralkoxycarbonyl, such as CBZ (“carbobenzoxy”), 4-methoxybenzyloxycarbonyl and FMOC; and arylsulfonyl, such as Mtr. Preferred aminoprotecting groups are BOC and Mtr, further-more CBZ, Fmoc, benzyl and acetyl.

The BOC, OtBu and Mtr groups can, for example, preferably be cleaved off using TFA in dichloromethane or using approximately 3 to 5N HCl in dioxane at 15-30° C., and the FMOC group can be cleaved off using an approximately 5 to 50% solution of dimethylamine, diethylamine or piperidine in DMF at 15-30° C.

Protecting groups which can be removed hydrogenolytically (for example CBZ, benzyl or the liberation of the amidino group from the oxadiazole derivative thereof) can be cleaved off, for example, by treatment with hydrogen in the presence of a catalyst (for example a noble-metal catalyst, such as palladium, advantageously on a support, such as carbon).

Suitable solvents here are those indicated above, in particular, for example, alcohols, such as methanol or ethanol, or amides, such as DMF. The hydrogeno lysis is generally carried out at temperatures between about 0 and 100° C. and pressures between about 1 and 200 bar, preferably at 20-30° C. and 1-10 bar. Hydrogeno lysis of the CBZ group succeeds well, for example, on 5 to 10% Pd/C in methanol or using ammonium formate (instead of hydrogen) on Pd/C in methanol/DMF at 20-30° C.

It is also possible for a plurality of—identical or different—protected amino and/or hydroxyl groups to be present in the molecule of the starting material. If the protecting groups present are different from one another, they can in many cases be cleaved off selectively.

The compounds described herein are liberated from their functional derivatives-depending on the protecting group used—for example strong inorganic acids, such as hydrochloric acid, perchloric acid or sulfuric acid, strong organic carboxylic acids, such as trichloroacetic acid, TFA or sulfonic acids, such as benzene- or p-toluenesulfonic acid. The presence of an additional inert solvent is possible, but is not always necessary.

In some embodiments, oxidation of compound 1_1 can be accomplished using methods known to those of ordinary skill in the art. For example, oxidation of compound 1_1 may be accomplished using oxidizing agent which is Py·SO₃. In some embodiments, oxidation of compound 1_1 may be accomplished using Py·SO₃, TEA and DMSO. In some embodiments, oxidation of compound 1_1 may be accomplished using Py·SO₃, TEA, and DMSO, in DCM.

In some embodiments, esterification of compound 3A can be accomplished by treating compound 3 with an azodicarboxylate. In some embodiments, an azodicarboxylate is DEAD or DIAD. In some embodiments, azodicarboxylate is DEAD.

In some embodiments, hydroboration-oxidation of compound 4 can be accomplished by treating compound 4 with BH₃ followed by an oxidative work-up, for example, NaBO₃.

In some embodiments, compound 6 can be prepared by treating compound 5 with an azodicarboxylate and compound 5A. In some embodiments, an azodicarboxylate is DEAD or DIAD. In some embodiments, azodicarboxylate is DEAD.

In some embodiments, LG is selected from the group consisting of halogen, —OTf, —OMs, and —OTs. In some embodiments, LG is selected from the group consisting of —OMs.

Suitable inert solvents are preferably organic, for example carboxylic acids, such as acetic acid, ethers, such as tetrahydrofuran or dioxane, amides, such as DMF, halogenated hydrocarbons, such as dichloromethane, furthermore also alcohols, such as methanol, ethanol or isopropanol, and water. Mixtures of the above-mentioned solvents are furthermore suitable. TFA is preferably used in excess without addition of a further solvent, and perchloric acid is preferably used in the form of a mixture of acetic acid and 70% perchloric acid in the ratio 9:1. The reaction temperatures for the cleavage are advantageously between about 0 and about 50° C., preferably between 15 and 30° C. (room temperature).

Examples of suitable inert solvents are hydrocarbons, such as hexane, petroleum ether, benzene, toluene or xylene; chlorinated hydrocarbons, such as trichloroethylene, 1,2-dichloroethane, tetrachloromethane, trifluoromethylbenzene, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide, N-methylpyrrolidone (NMP) or dimethyl-formamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents.

Esters can be hydrolyzed, for example, using HC1, H₂SO₄, or using LiOH, NaOH or KOH in water, water/THF, water/THF/ethanol or water/dioxane, at temperatures between 0 and 100° C.

Free amino groups can furthermore be acylated in a conventional manner using an acyl chloride or anhydride or alkylated using an unsubstituted or substituted alkyl halide, advantageously in an inert solvent, such as dichloromethane or THF and/or in the presence of a base, such as triethylamine or pyridine, at temperatures between −60° C. and +30° C.

For all the protection and deprotection methods, see Philip J. Kocienski, in “Protecting Groups”, Georg Thieme Verlag Stuttgart, New York, 1994 and, Theodora W. Greene and Peter G. M. Wuts in “Protective Groups in Organic Synthesis”, Wiley Interscience, 3^{rd} Edition 1999.

Reaction schemes as described in the example section are illustrative only and should not be construed as limiting the disclosure in any way.

In some embodiments, compound (R)-11 is at least 80%, at least 90%, at least 95%, at least 99%, or at least 99.9% enantiomerically pure.

### ENUMERATED EMBODIMENTS

The present disclosure includes the enumerated embodiments 1-84:

1. A process for preparing compound (R)-11, or a pharmaceutically acceptable salt or solvate thereof,

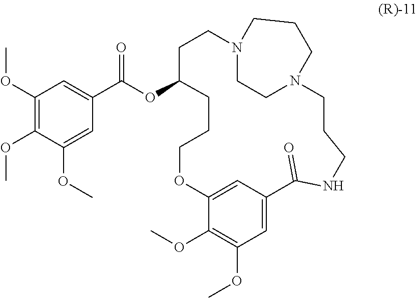 [SMILES: COc1cc(C(=O)O[C@@H]2CCCOc3cc(cc(OC)c3OC)C(=O)NCCCN3CCCN(CC2)CC3)cc(OC)c1OC]

comprising the step of separating compound (R)-11 from a racemic mixture of compound 11:

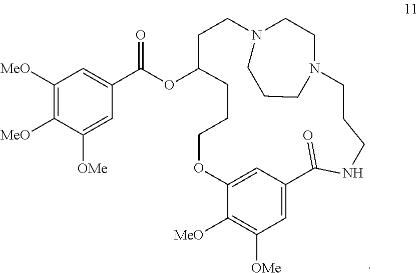 [SMILES: COc1cc(C(=O)OC2CCCOc3cc(cc(OC)c3OC)C(=O)NCCCN3CCCN(CC2)CC3)cc(OC)c1OC]

2. The process of embodiment 1, wherein the step of separating compound (R)-11 is accomplished using chiral supercritical fluid chromatography (chiral-SFC).

3. The process of any of embodiments 1-2, further comprising the step of reacting compound 10 with a peptide coupling reagent to prepare compound 11:

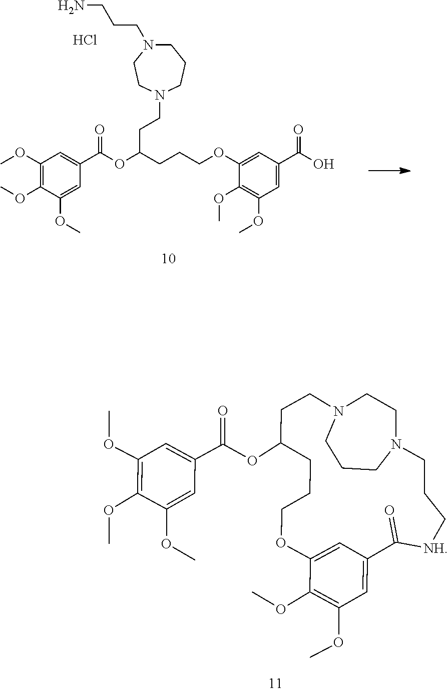 [SMILES: COc1cc(C(=O)OC(CCCOc2cc(C(=O)O)cc(OC)c2OC)CCN2CCCN(CCCN)CC2)cc(OC)c1OC ; Cl ; COc1cc(C(=O)OC2CCCOc3cc(cc(OC)c3OC)C(=O)NCCCN3CCCN(CC2)CC3)cc(OC)c1OC]

4. The process of embodiment 3, wherein the peptide coupling reagent selected from the group consisting of BOP, PyBOP, HATU, and HBTU.

5. The process of any of embodiments 3-4, wherein the peptide coupling reagent is PyBOP.

6. The process of any of embodiments 3-5, further comprising the step of deprotecting compound 9 to prepare compound 10 by reacting compound 9 with an acid:

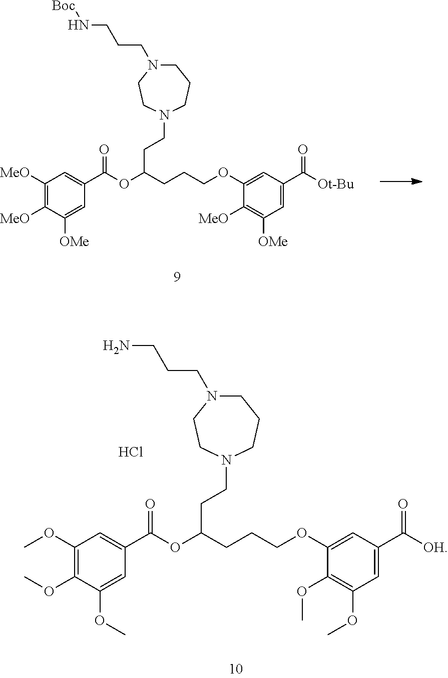 [SMILES: COc1cc(C(=O)OC(CCCOc2cc(C(=O)OC(C)(C)C)cc(OC)c2OC)CCN2CCCN(CCCNC(=O)OC(C)(C)C)CC2)cc(OC)c1OC ; COc1cc(C(=O)OC(CCCOc2cc(C(=O)O)cc(OC)c2OC)CCN2CCCN(CCCN)CC2)cc(OC)c1OC ; Cl]

7. The process of embodiment 6, wherein the acid is HCl.

8. The process of any of embodiments 5-6, further comprising the step of reacting compound 8 with compound 8A to prepare compound 9:

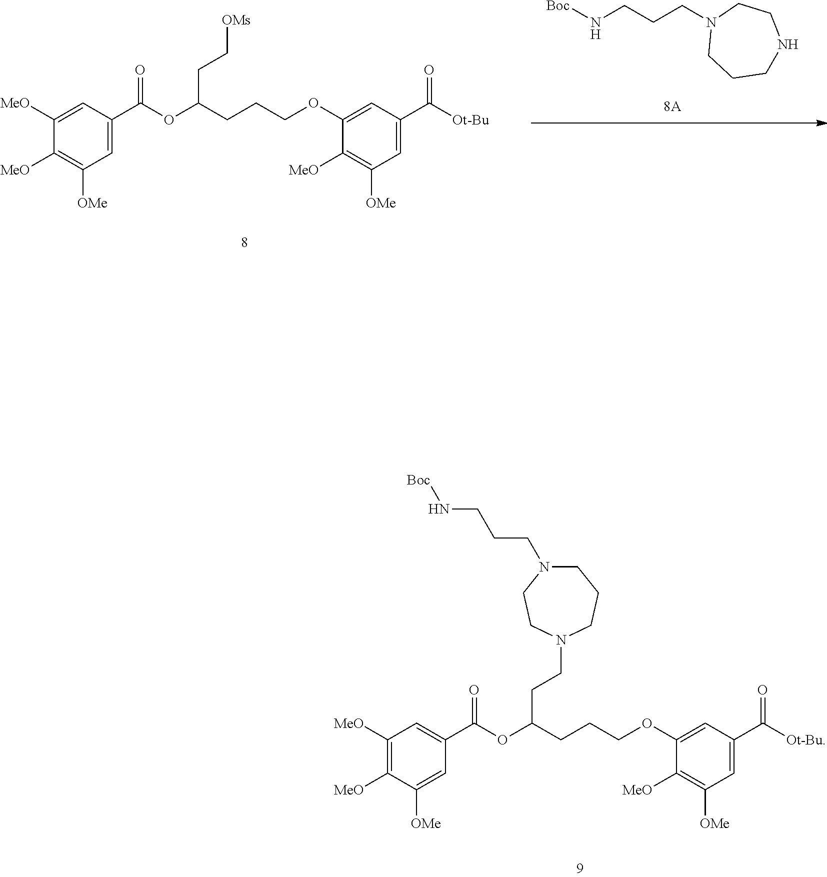 [SMILES: COc1cc(C(=O)OC(CCCOc2cc(C(=O)OC(C)(C)C)cc(OC)c2OC)CCOS(C)(=O)=O)cc(OC)c1OC ; CC(C)(C)OC(=O)NCCCN1CCCNCC1 ; COc1cc(C(=O)OC(CCCOc2cc(C(=O)OC(C)(C)C)cc(OC)c2OC)CCN2CCCN(CCCNC(=O)OC(C)(C)C)CC2)cc(OC)c1OC]

9. The process of embodiment 8, wherein the step of reacting compound 8 with compound 8A comprises addition of a suitable base.

10. The process of embodiment 9, wherein the suitable base is selected from the group consisting of K₂CO₃, Na₂CO₃, and Ca₂CO₃.

11. The process of embodiment 10, wherein the suitable base is K₂CO₃.

12. The process of any of embodiments 8-11, further comprising the step of reacting compound 7 with a mesylating agent to prepare compound 8:

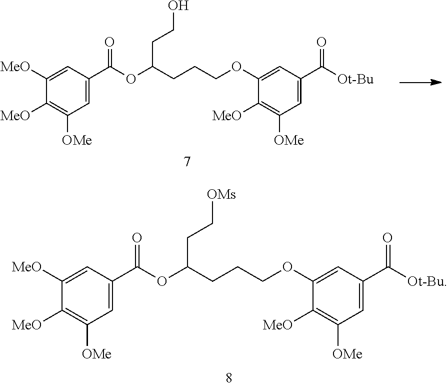 [SMILES: COc1cc(C(=O)OC(CCO)CCCOc2cc(C(=O)OC(C)(C)C)cc(OC)c2OC)cc(OC)c1OC ; COc1cc(C(=O)OC(CCCOc2cc(C(=O)OC(C)(C)C)cc(OC)c2OC)CCOS(C)(=O)=O)cc(OC)c1OC]

13. The process of embodiment 12, wherein the mesylating agent is MsCl.

14. The process of any of embodiments 12-13, wherein the step of reacting compound 7 with a mesylating agent comprises the addition of a suitable base.

15. The process of embodiment 14, wherein the suitable base is selected from the group consisting of TEA, DEA, DIPA, and pyridine.

16. The process of embodiment 15, wherein the suitable base is TEA.

17. The process of any of embodiments 12-16, further comprising reacting compound 6 with a suitable deprotecting agent to prepare compound 7:

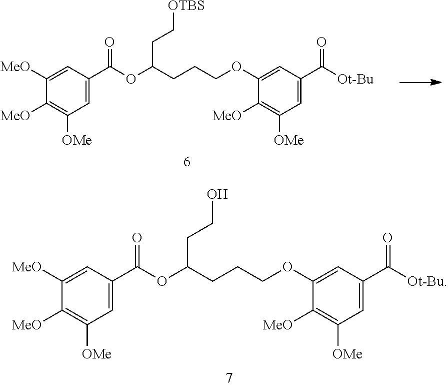 [SMILES: COc1cc(C(=O)OC(CCCOc2cc(C(=O)OC(C)(C)C)cc(OC)c2OC)CCO[Si](C)(C)C(C)(C)C)cc(OC)c1OC ; COc1cc(C(=O)OC(CCO)CCCOc2cc(C(=O)OC(C)(C)C)cc(OC)c2OC)cc(OC)c1OC]

18. The process of embodiment 17, wherein the suitable deprotecting agent is a fluoride source.

19. The process of embodiment 18, wherein the fluoride source is selected from HF-pyridine, TBAF, KF, and TBAT.

20. The process of embodiment 19, wherein the fluoride source is HF-pyridine.

21. The process of any of embodiments 17-20, further comprising the step of reacting compound 5 with compound 5A to prepare compound 6

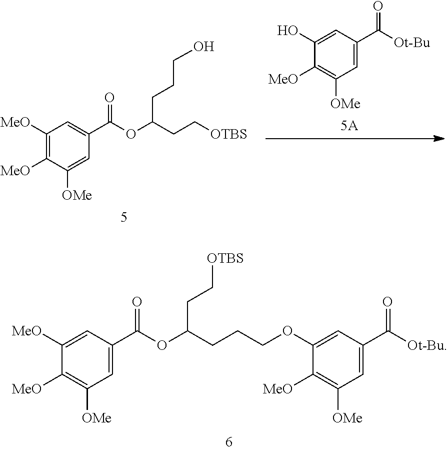 [SMILES: COc1cc(C(=O)OC(CCCO)CCO[Si](C)(C)C(C)(C)C)cc(OC)c1OC ; COc1cc(C(=O)OC(C)(C)C)cc(O)c1OC ; COc1cc(C(=O)OC(CCCOc2cc(C(=O)OC(C)(C)C)cc(OC)c2OC)CCO[Si](C)(C)C(C)(C)C)cc(OC)c1OC]

22. The process of embodiment 21, wherein the step of reacting compound 5 with compound 5A further comprises addition of DEAD and PPh₃.

23. The process of any of embodiments 21-22, further comprising the step of compound 5 from compound 4:

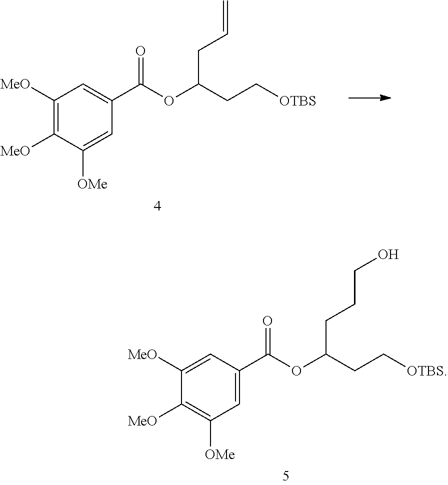 [SMILES: C=CCC(CCO[Si](C)(C)C(C)(C)C)OC(=O)c1cc(OC)c(OC)c(OC)c1 ; COc1cc(C(=O)OC(CCCO)CCO[Si](C)(C)C(C)(C)C)cc(OC)c1OC]

24. The process of embodiment 23, wherein the step of compound 5 from compound 4 comprises a hydroboration-oxidation reaction sequence.

25. The process of embodiment 24, wherein the hydroboration-oxidation reaction sequence comprises the steps of (a) addition of BH₃/THF; (b) quenching with H₂O (c) addition of NaBO₃.

26. The process of any of embodiments 23-25, further comprising the step of reacting compound 3 with compound 3A to prepare compound 4:

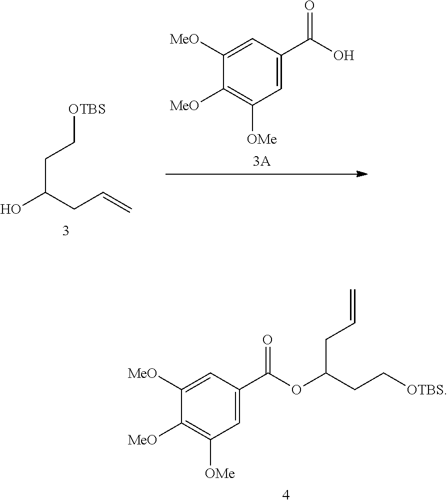 [SMILES: C=CCC(O)CCO[Si](C)(C)C(C)(C)C ; COc1cc(C(=O)O)cc(OC)c1OC ; C=CCC(CCO[Si](C)(C)C(C)(C)C)OC(=O)c1cc(OC)c(OC)c(OC)c1]

27. The process of embodiment 26, wherein the step of reacting compound 3 with compound 3A comprises addition of an ester coupling reagent.

28. The process of embodiment 27, wherein the ester coupling reagent is DCC.

29. The process of any of embodiments 26-28, further comprising the step of reacting compound 1 with compound 2 to prepare compound 3:

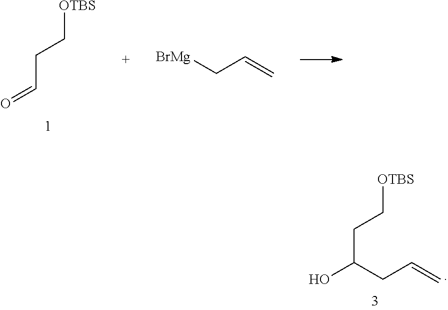

30. The process of embodiment 29, further comprising the step of oxidizing compound 1_1 to prepare compound 1:

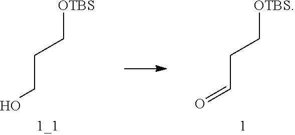 [SMILES: CC(C)(C)[Si](C)(C)OCCCO ; CC(C)(C)[Si](C)(C)OCCC=O]

31. The process of embodiment 30, wherein the step of oxidizing compound 1_1 comprises addition of an oxidizing agent.

32. The process of embodiment 31, wherein the oxidizing agent is Py·SO₃.

33. A process of preparing a compound (R)-11 comprising the step of reacting compound 18 with compound 3A:

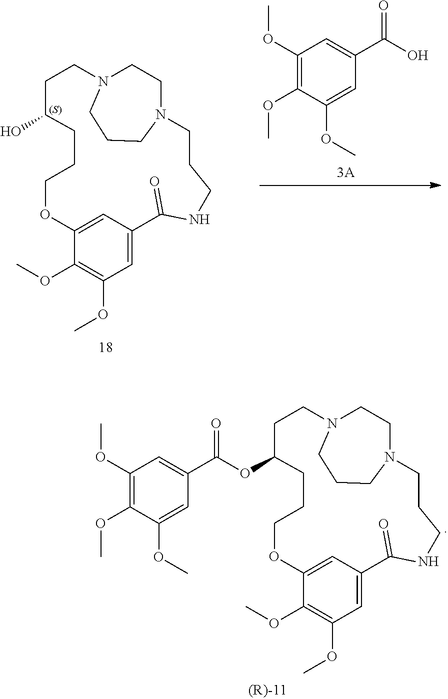

34. The process of embodiment 33, wherein the step of reacting compound 18 with compound 3A comprises addition of an azodicarboxylate.

35. The process of embodiment 34, wherein the azodicarboxylate is DEAD or DIAD.

36. The process of embodiment 35, wherein the azodicarboxylate is DEAD.

37. The process of any of embodiments 34-36, wherein the step of reacting compound 18 with compound 3A further comprises addition of PPh₃.

38. A process for preparing compound (R)-11, or a pharmaceutically acceptable salt or solvate thereof,

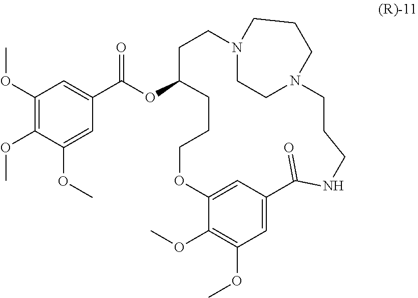 [SMILES: COc1cc(C(=O)O[C@@H]2CCCOc3cc(cc(OC)c3OC)C(=O)NCCCN3CCCN(CC2)CC3)cc(OC)c1OC]

comprising the step of reacting compound (R)-10 with a peptide coupling reagent:

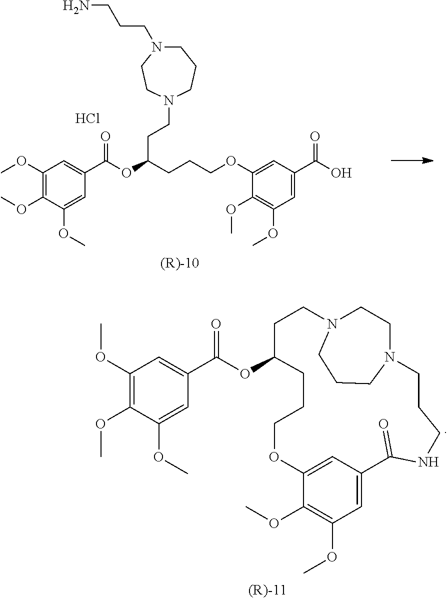 [SMILES: COc1cc(C(=O)O[C@H](CCCOc2cc(C(=O)O)cc(OC)c2OC)CCN2CCCN(CCCN)CC2)cc(OC)c1OC ; Cl ; COc1cc(C(=O)O[C@@H]2CCCOc3cc(cc(OC)c3OC)C(=O)NCCCN3CCCN(CC2)CC3)cc(OC)c1OC]

39. The process of embodiment 38, wherein the peptide coupling reagent selected from the group consisting of BOP, PyBOP, HATU, and HBTU.

40. The process of any of embodiments 38-39, wherein the peptide coupling reagent is PyBOP.

41. The process of any of embodiments 38-40, further comprising the step of deprotecting compound 9 to prepare compound (R)-10 by reacting compound (R)-9 with an acid:

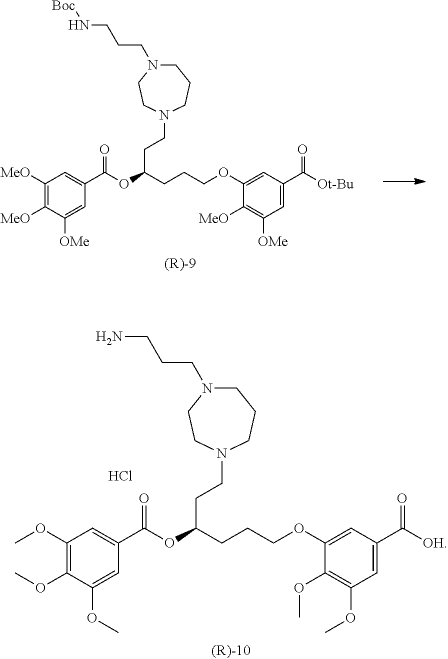 [SMILES: COc1cc(C(=O)O[C@H](CCCOc2cc(C(=O)OC(C)(C)C)cc(OC)c2OC)CCN2CCCN(CCCNC(=O)OC(C)(C)C)CC2)cc(OC)c1OC ; COc1cc(C(=O)O[C@H](CCCOc2cc(C(=O)O)cc(OC)c2OC)CCN2CCCN(CCCN)CC2)cc(OC)c1OC ; Cl]

42. The process of embodiment 41, wherein the acid is HCl.

43. The process of any of embodiments 38-42, further comprising the step of reacting compound (R)-17 with 3A to prepare compound (R)-9:

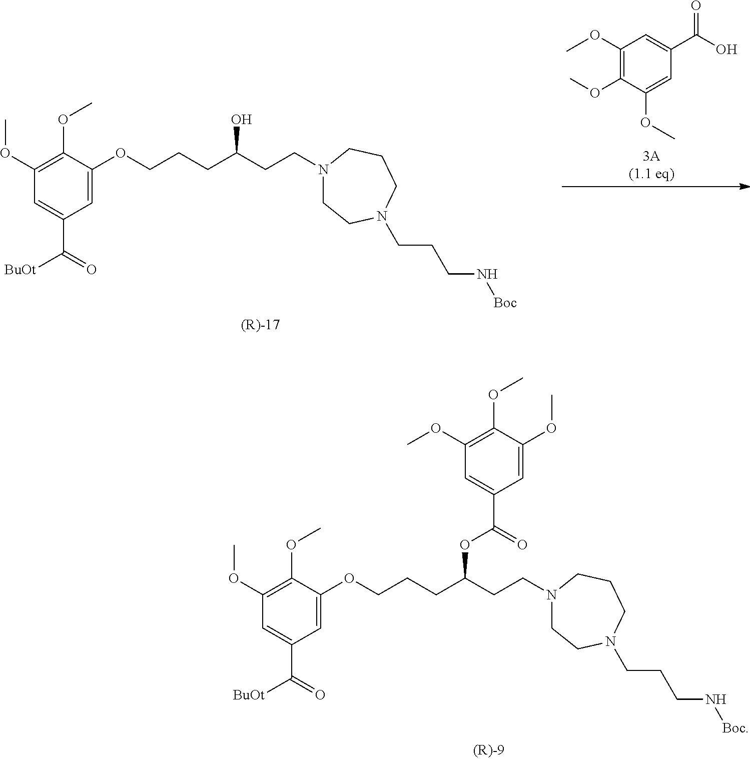

44. The process of embodiment 43, wherein the step of reacting compound (R)-17 with compound 3A comprises addition of a carbodiimide.

45. The process of embodiment 44, wherein the carbodiimide is selected from the group consisting of DIC and DCC.

46. The process of embodiment 45, wherein the carbodiimide is DIC.

47. The process of any of embodiments 43-46, further comprising a preliminary step of increasing the enantiomeric purity of compound 17 using a resolving agent.

48. The process of embodiment 47, wherein the resolving agent is

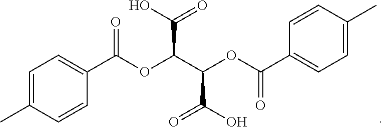

49. The process of any of embodiments 43-48, further comprising reacting compound 15 with a suitable reducing agent to prepare compound (R)-17:

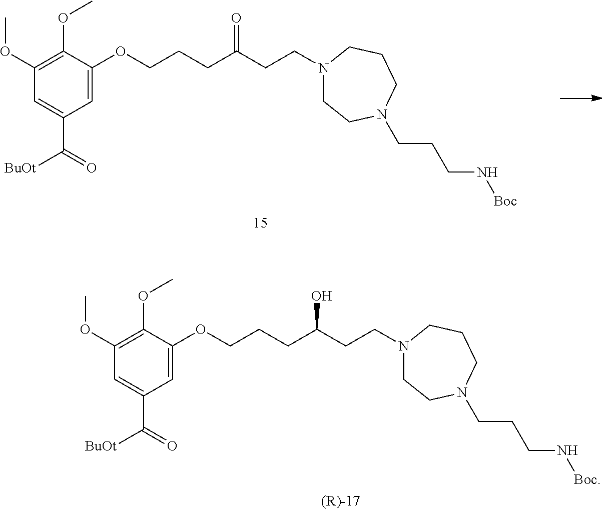

50. The process of embodiment 49, wherein the suitable reducing agent is an enantiomeric reducing agent.

51. The process of embodiment 50, where the enantiomeric reducing agent is (S,S)-Ms-DENEB.

52. The process of any of embodiments 49-51, further comprising the step of reacting compound 14 with compound 8A to prepare compound 15:

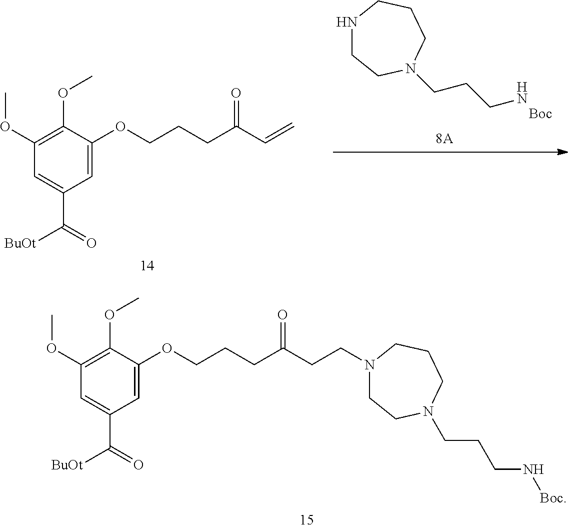

53. The process of embodiment 52, wherein the step of reacting compound 14 with compound 8A comprises addition of a suitable base.

54. The process of embodiment 53, wherein the suitable base is triethylamine.

55. The process of any of embodiments 53-54, further comprising the step of preparing compound 14 from compound 13:

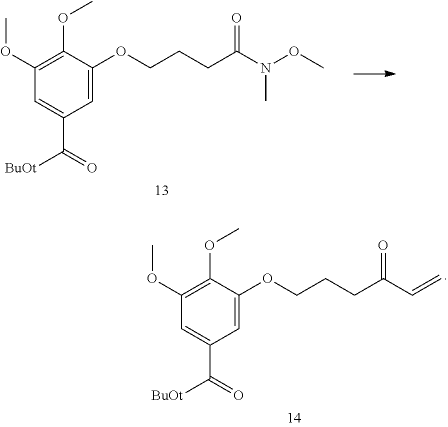

56. The process of embodiment 55, further comprising the step of reacting compound 12 with compound 5A to prepare compound 13:

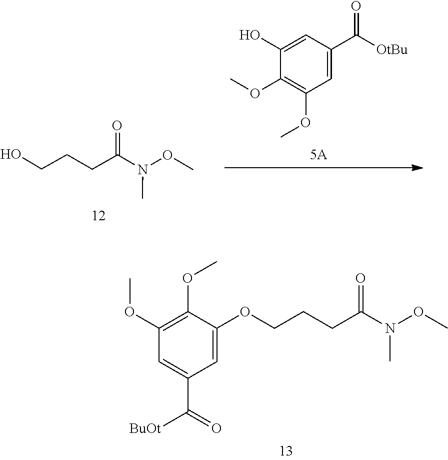

57. The process of embodiment 56, wherein the step of reacting compound 12 with compound 5A comprises addition of an azodicarboxylate.

58. The process of embodiment 57, wherein the azodicarboxylate is DEAD.

59. The process of any of embodiments 56-58, further comprising the step of reacting compound 12B with compound 12A to prepare compound 12:

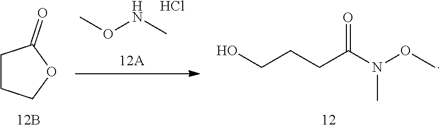

60. The process of embodiment 59, wherein the step of reacting compound 11 with compound 11A comprises addition of a reducing agent.

61. The process of embodiment 60, wherein the reducing agent is DIBAL-H.

62. A process for preparing compound (R)-11, or a pharmaceutically acceptable salt or solvate thereof,

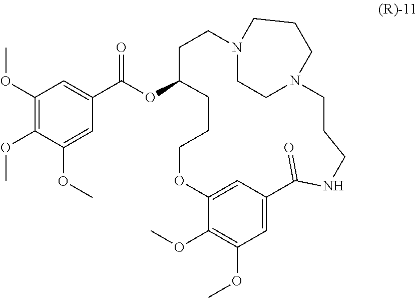 [SMILES: COc1cc(C(=O)O[C@@H]2CCCOc3cc(cc(OC)c3OC)C(=O)NCCCN3CCCN(CC2)CC3)cc(OC)c1OC]

comprising the step of reacting compound (R)-10 with a peptide coupling reagent:

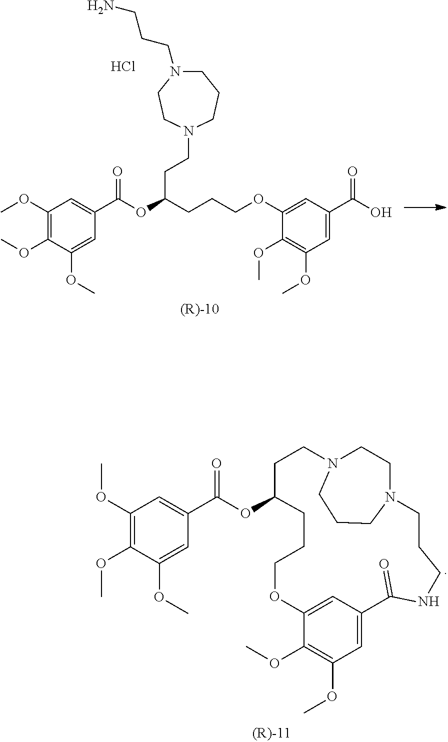 [SMILES: COc1cc(C(=O)O[C@H](CCCOc2cc(C(=O)O)cc(OC)c2OC)CCN2CCCN(CCCN)CC2)cc(OC)c1OC ; Cl ; COc1cc(C(=O)O[C@@H]2CCCOc3cc(cc(OC)c3OC)C(=O)NCCCN3CCCN(CC2)CC3)cc(OC)c1OC]

63. The process of embodiment 62, wherein the peptide coupling reagent selected from the group consisting of BOP, PyBOP, HATU, and HBTU.

64. The process of any of embodiments 62-63, wherein the peptide coupling reagent is PyBOP.

65. The process of any of embodiments 62-64, further comprising the step of deprotecting compound (R)-9 to prepare compound (R)-10 by reacting compound (R)-17 with an acid:

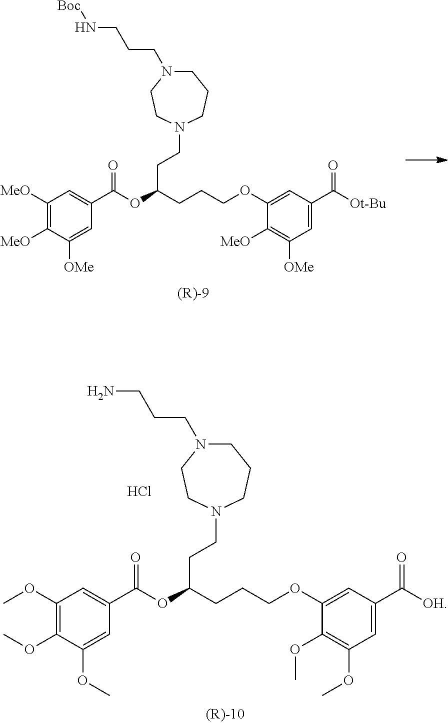 [SMILES: COc1cc(C(=O)O[C@H](CCCOc2cc(C(=O)OC(C)(C)C)cc(OC)c2OC)CCN2CCCN(CCCNC(=O)OC(C)(C)C)CC2)cc(OC)c1OC ; COc1cc(C(=O)O[C@H](CCCOc2cc(C(=O)O)cc(OC)c2OC)CCN2CCCN(CCCN)CC2)cc(OC)c1OC ; Cl]

66. The process of embodiment 65, wherein the acid is HCl.

67. The process of any of embodiments 62-66, further comprising the step of reacting compound 24 with compound 25A to prepare compound (R)-9:

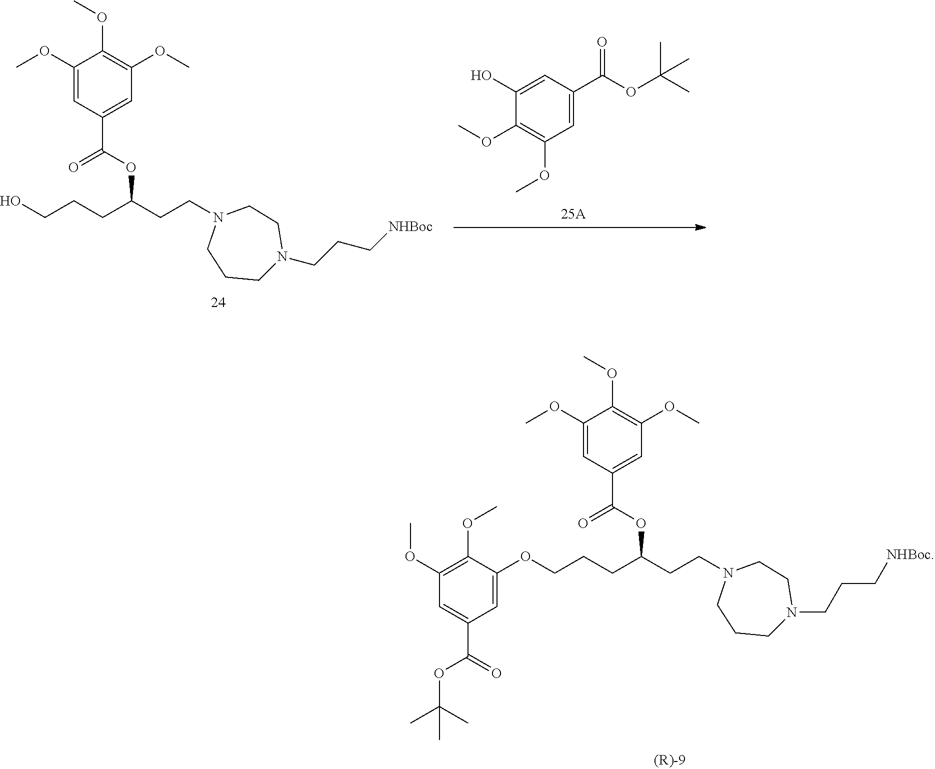 [SMILES: COc1cc(C(=O)O[C@H](CCCO)CCN2CCCN(CCCNC(=O)OC(C)(C)C)CC2)cc(OC)c1OC ; COc1cc(C(=O)OC(C)(C)C)cc(O)c1OC ; COc1cc(C(=O)O[C@H](CCCOc2cc(C(=O)OC(C)(C)C)cc(OC)c2OC)CCN2CCCN(CCCNC(=O)OC(C)(C)C)CC2)cc(OC)c1OC]

68. The process of embodiment 67, wherein the step of reacting compound 24 with compound 25A comprises addition of an azodicarboxylate.

69. The process of embodiment 68, wherein the azodicarboxylate is selected from the group consisting of DEAD and DIAD.

70. The process of embodiment 69, wherein the azodicarboxylate is DEAD.

71. The process of any of embodiments 67-70, further comprising the step of reacting compound 23 with a reducing agent to prepare compound 24:

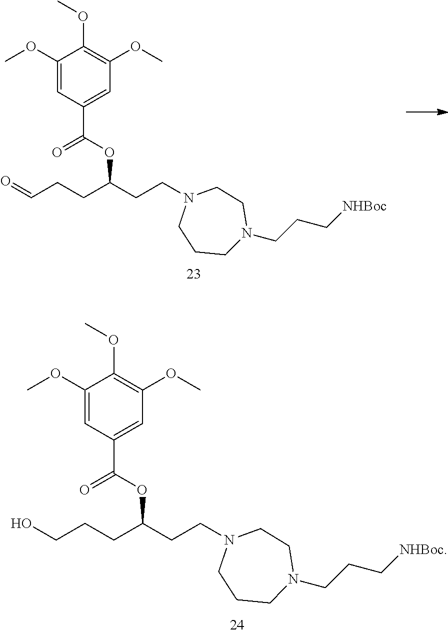 [SMILES: COc1cc(C(=O)O[C@H](CCC=O)CCN2CCCN(CCCNC(=O)OC(C)(C)C)CC2)cc(OC)c1OC ; COc1cc(C(=O)O[C@H](CCCO)CCN2CCCN(CCCNC(=O)OC(C)(C)C)CC2)cc(OC)c1OC]

72. The process of embodiment 71, wherein the reducing agent is NaBH₄.

73. The process of any of embodiments 71-72, further comprising the step of reacting compound 22 with a catalyst to prepare compound 23:

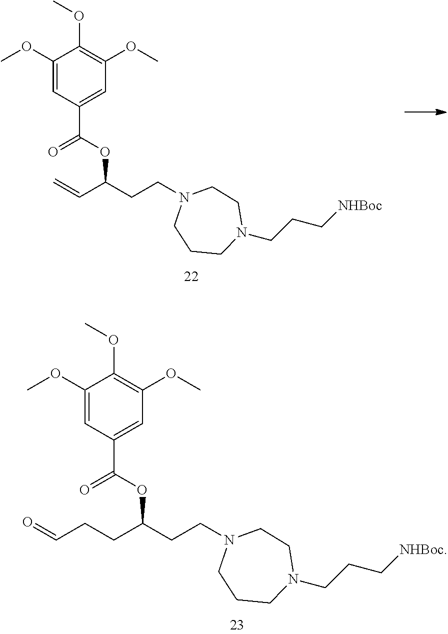 [SMILES: C=C[C@H](CCN1CCCN(CCCNC(=O)OC(C)(C)C)CC1)OC(=O)c1cc(OC)c(OC)c(OC)c1 ; COc1cc(C(=O)O[C@H](CCC=O)CCN2CCCN(CCCNC(=O)OC(C)(C)C)CC2)cc(OC)c1OC]

74. The process of embodiment 73, wherein the catalyst is RhCl(PPh₃)₃.

75. The process of any of embodiments 73-74, further comprising the step of reacting compound 21 with compound 3A to prepare compound 22:

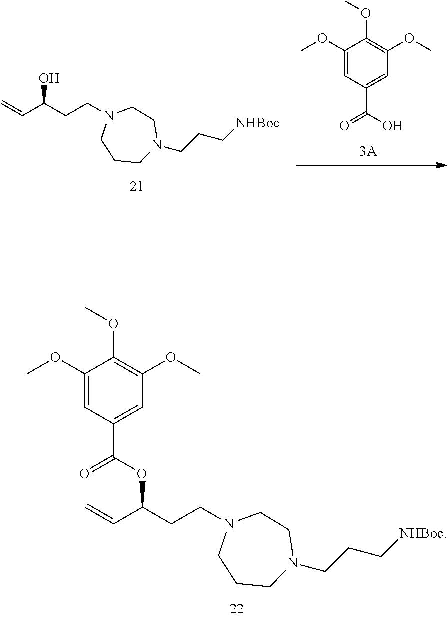

76. The process of embodiment 75, wherein the step of reacting compound 21 with compound 3A comprises addition of an ester coupling reagent.

77. The process of embodiment 76, wherein the ester coupling reagent is DCC.

78. The process of any of embodiments 75-77, further comprising the step of reacting compound 20 with compound 8A to prepare compound 21:

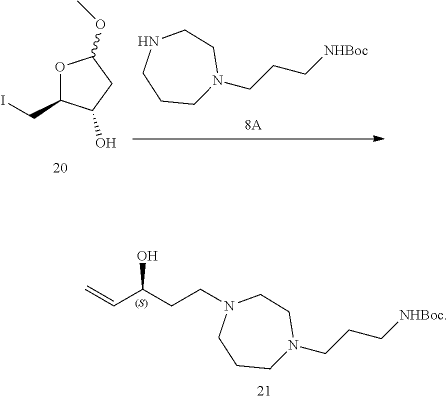

79. The process of embodiment 78, wherein the step of reacting compound 20 with compound 8A comprises addition of a suitable reducing agent.

80. The process of embodiment 79, wherein the reducing agent is NaBH₄.

81. The process of any of embodiments 78-80, further comprising the step of preparing compound 20 from compound 19:

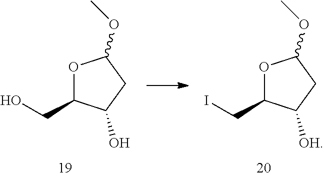

82. The process of embodiment 81, wherein the step of preparing compound 20 comprises addition of PPh₃, I₂, and imidazole.

83. The process of any of embodiments 81-82, further comprising the step of preparing compound 19 from compound 18:

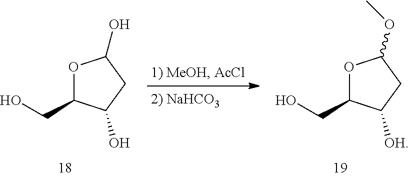

84. A process of preparing a compound (R)-11 comprising the step of reacting compound 18 with compound 3A:

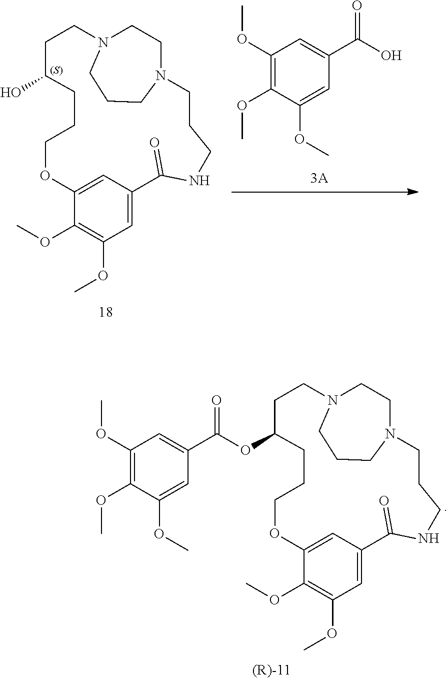

### Exemplification

The present invention will be better understood with reference to the following examples. These examples are intended to representative of specific embodiments of the invention, and are not intended as limiting the scope of the invention.

The following abbreviations are used:
MeOH: Methanol
THF: tetrahydrofuran;
DCM: dichloromethane;
EtOAC: ethyl acetate;
ACN: acetonitrile;
Et₃N: triethylamine;
DIPEA: N,N-Diisopropylethylamine;
N₂: nitrogen gas;
min: minute;
hr: hour;
Na₂SO₄: sodium sulfate;
MgSO₄: magnesium sulfate
prep-HPLC: preparative High-Pressure Liquid Chromatography;
HPLC: High Pressure Liquid Chromatography;
SiO₂: silica gel;
K₂CO₃: potassium carbonate;
LiOH: lithium hydroxide.
DEAD: Diethyl azodicarboxylate
PPh₃: triphenylphosphine
OPPh₃: triphenymphosphine oxyde
TFA: trifluoroacetic acid
PE/EA: Petrol ether/Ethyl acetate
CHCl₃: chloroform
DCM: dichloromethane
MPLC: Medium pressure liquid chromatography
Pd/C: palladium on charcoal
DMSO: dimethylsulfoxyde
Py·SO₃: Sulfur trioxide pyridine complex
DiBAl-H: Diisobutylaluminum hydride
NaHCO₃: Sodium bicarbonate
BH₃. THF: Borane tetrahydrofuran complex
NaBO₃: Sodium Perborate
HCOOH: formic acid
MEK: methyl ethyl ketone
DIC: N,N′-Diisopropylcarbodiimide
(S,S)-Ms-DENEB: Chloro[(S,S)—N-[2-(4-methylbenzyloxy)ethyl]-N′-(p-toluenesulfonyl)-1,2-diphenylethylenediamine]ruthenium (II)
BOP: benzotriazol-1-yloxytris(dimethylamino)phosphonium hexafluorophosphate
PyBOP: benzotriazol-1-yloxytripyrrolidinophosphonium hexafluorophosphate
HATU: 1-[bis(dimethylamino)methylene]-1H-1,2,3-triazolo[4,5-b]pyridinium 3-oxide hexafluorophosphate
HBTU: (2-(1H-benzotriazol-1-yl)-1,1,3,3-tetramethyluronium hexafluorophosphate
MsCl: methanesulfonyl chloride
TEA: triethylamine
DEA: diethylamine
DIPA: diisopropylamine
TBAF: tetrabutylammonium fluoride
TBAT: tetrabutylammonium difluorotriphenylsilicate
DIAD: diisopropyl azodicarboxylate
DCC: dicyclohexylcarbodiimide

### I. CHEMISTRY EXAMPLES

### LCMS:

### Method 1:

| Instrument: | Agilent 1200 HPLC MSD:6120 single quadrupole MSD | | | | | | |
|---|---|---|---|---|---|---|---|
| Column: | Luna C18, 2.0*50 mm, 5 μm | | | | | | |
| Column | 40° C. | | | | | | |
| temperature: | | | | | | | |
| Mobile phase | 0.04% TFA in H2O | | | | | | |
| A(MPA) | | | | | | | |
| Mobile phase | 0.02% TFA in ACN | | | | | | |
| B(MPB) | | | | | | | |
| Flow rate: | 1.0 ml/min | | | | | | |
| | Time(min) | 0.01 | 0.40 | 3.00 | 4.00 | 4.01 | 4.50 |
| Gradient | MPA(%) | 95 | 95 | 5 | 5 | 95 | 95 |
| Ratio: | MPB(%) | 5 | 5 | 95 | 95 | 5 | 5 |

| | |
|---|---|
| Detection: | 220 nm |

### Method 2:

Instrument: Shimadzu LC-20AD MSD: LCMS-2020
Column: Kinetex 5 um EVO C18 30*2.1 mm
Column Temp: 40
Mobile Phase: A: 0.04% TFA in H2O
Mobile Phase: B: 0.02% TFA in ACN
Flow Rate: 1.5 ml/min

| Time | B % | Flow(ml/min) |
|---|---|---|
| 0.01 | 5 | 1.5 |
| 0.70 | 95 | 1.5 |
| 1.16 | 95 | 1.5 |
| 1.50 | 5 | 1.5 |

### Chirel HPLC:

Method 1:
Instrument: CAS-TJ-Chiral HPLC-K (Waters Arc with PDA detector)
Proc. Chnl. Descr.: 2998 PDA 254.0 nm (2998 (190-300) nm)
Column: Chiralpak IC-3, 50×4.6 mm, I.D., 3 μm
Mobile phase: A: Heptane B: EtOH (0.05% DEA, v/v)
Gradient: A:B=20:80
Flow rate: 1 mL/min
Column temp.: 35° C.

### Method 2

Instrument: CAS-TJ-ANA-Chiral HPLC-K (Waters Arc with 2998)
Proc. Chnl. Descr.: 2998 PDA 254.0 nm (2998 (190-300) nm)
Column: Chiralpak IF-3, 150×4.6 mm I.D., 3 μm
Mobile phase: A: Hexane B:EtOH+ACN (4:1) (0.05% IPAm, v/v)
Gradient: A:B=92:8
Flow rate: 1 mL/min
Column temp.: 30° C.

### NMR Analysis

The NMR data provided in the examples described below were obtained as followed:
1H-NMR: Bruker DPX 400 MHz. Abbreviations for multiplicities observed in NMR spectra are as follows: s (singlet), d (doublet), t (triplet), q (quadruplet), m (multiplet), br (broad). Solvents, reagents and starting materials were purchased and used as received from commercial vendors unless otherwise specified.

### Intermediate Compound 5A

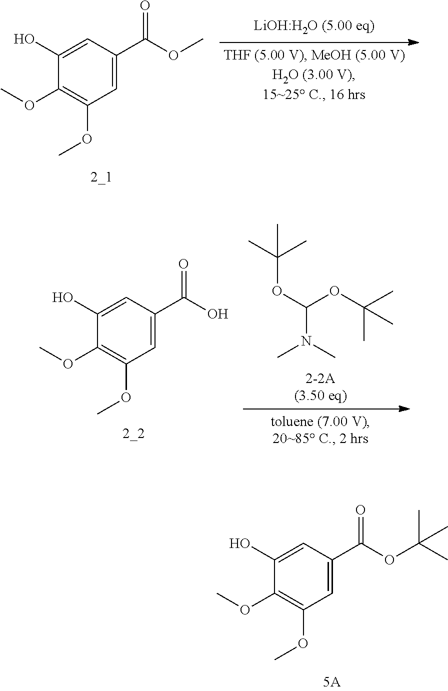

At 15-25° C., THF (4.80 L), MeOH (1.60 L, 1.00V), H₂O (1.60 L, 1.00V), 2_1 (1.60 kg, 7.56 mol, 1.0 eq) were charged into reactor at 15˜25° C. Then, LiOH·H₂O (1.58 kg, 37.7 mol, 5.0 eq) were charged with 5 portions into reactor. The reaction was keeping stirring at 30˜35° C. for 16 hours. An aqueous solution of HCl (3 M) was added dropwise into the mixture at 15˜25° C. until the pH=3˜4. The organic phase was separated and the aqueous layer was extracted with ethyl acetate (2.00 L×2). The combined organic phase was washed with brine (2.00 L), dry over Na₂SO₄, filtered and concentrated under reduced pressure to give the product. The compound 2_2 (1.30 kg, 69.6% yield, 98.9% purity) is obtained as white solid.

1H NMR (400 MHZ, DMSO-d₆) δ 12.7 (brs, 1H), 9.52 (brs, 1H), 7.10 (s, 1H), 7.03 (s, 1H), 3.80 (s, 3H), 3.72 (s, 3H)

At 20-25° C., toluene (7.00 L), compound 2_2 (1.00 kg, 4.94 mol, 1.00 eq) were charged into the reactor and heated to 80-85° C. The compound 2-2A (3.59 kg, 17.6 mol, 3.50 eq) were added with 5 portions into the reactor at 80-85° C. The reaction mixture was stirred at 80-85° C. for 16 hrs. The reaction mixture was concentrated to give a residue. The residue was purified by column chromatography (SiO₂, n-hexane/ethyl acetate=40/1 to 20/1) to give the compound 5A (1.00 kg, 60.2% yield, 99.5% purity) as colorless oil.

1H NMR (400 MHZ, CDCl3-d) δ 7.18 (s, 1H), 7.09 (s, 1H), 5.81 (s, 1H), 3.87 (s, 3H), 3.82 (s, 3H), 1.50 (s, 9H).

### Intermediate Compound 8A

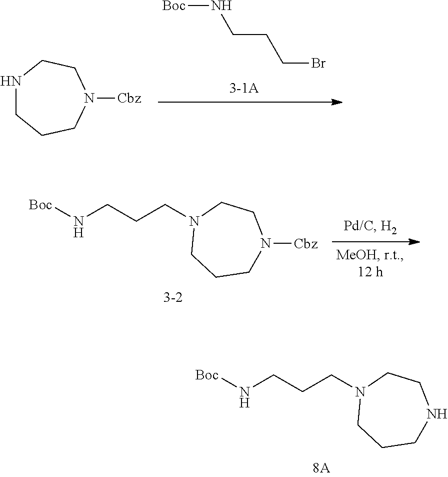 [SMILES: O=C(OCc1ccccc1)N1CCCNCC1 ; CC(C)(C)OC(=O)NCCCBr ; CC(C)(C)OC(=O)NCCCN1CCCN(C(=O)OCc2ccccc2)CC1 ; CC(C)(C)OC(=O)NCCCN1CCCNCC1]

At 15-25° C., acetonitrile (2.83 L, 10.0 V) compound 3-1A (373 g, 1.57 mol, 1.30 eq), KI (40.0 g, 0.23 mol, 0.20 eq), DIPEA (311 g, 2.41 mol, 2.0 eq), compound 3-1 (283 g, 1.21 mol, 1.00 eq) were charged into reactor. The reaction mixture was stirred at 70˜80° C. for 12 hrs. The reaction mixture was concentrated at 40˜45° C. H₂O (1.00 L) and EtOAc (1.00 L) were added into the mixture and stir at 15-25° C. for 10 mins. The organic phase was separated and washed with brine (1.00 L), dry over with Na₂SO₄ and concentrated to get the residue as yellow oil. The residue was purified by reversed-phase MPLC (0.10% NH₄OH in water and ACN) to afford the Compound 3-2 (890 g, 76.0% yield, 96.5% purity) as yellow oil.

1H NMR (400 MHZ, CDCl3-d) δ 7.28-7.38 (m, 5H), 5.42-5.47 (m, 1H), 5.14 (s, 2H), 3.51-3.58 (m, 4H), 3.18 (brs, 2H), 2.49-2.67 (m, 6H), 1.79-2.04 (m, 2H), 1.59-1.63 (m, 2H), 1.43 (s, 9H).

At 15-25° C., Pd/C (5.04 g, 10% wt), MeOH (350 mL, 7.00 V), compound 3-2 (50.4 g, 0.12 mol, 1.00 eq) was charged under argon. The reaction mixture was degassed with H₂ for 3 times, then stirred at 35° C. for 16 hours under H₂ (45 Psi). The reaction mixture was filtrated. The filter cake was washed with MeOH (500 mL). The filtrate was concentrated to get the residue as yellow oil. The residue was triturated with ACN (1.00 L) at 15˜25° C. for 30 min. The mixture was filtered and to remove the undissolved solid and collect the filtrate. The filtrate was concentrated the filtrate to get the compound 4A (705 g, 85.8% yield, 81.1% purity) as a yellow oil.

1H NMR (400 MHZ, CDCl3-d) δ 5.79 (s, 1H), 3.18-3.19 (m, 3H), 2.92-2.97 (m, 3H), 2.50-2.69 (m, 6H), 1.75-1.81 (m, 2H), 1.57-1.64 (m, 2H), 1.42 (s, 9H)

### Synthesis of Final Compounds

### Example 1. Synthesis of Compound (R)-11

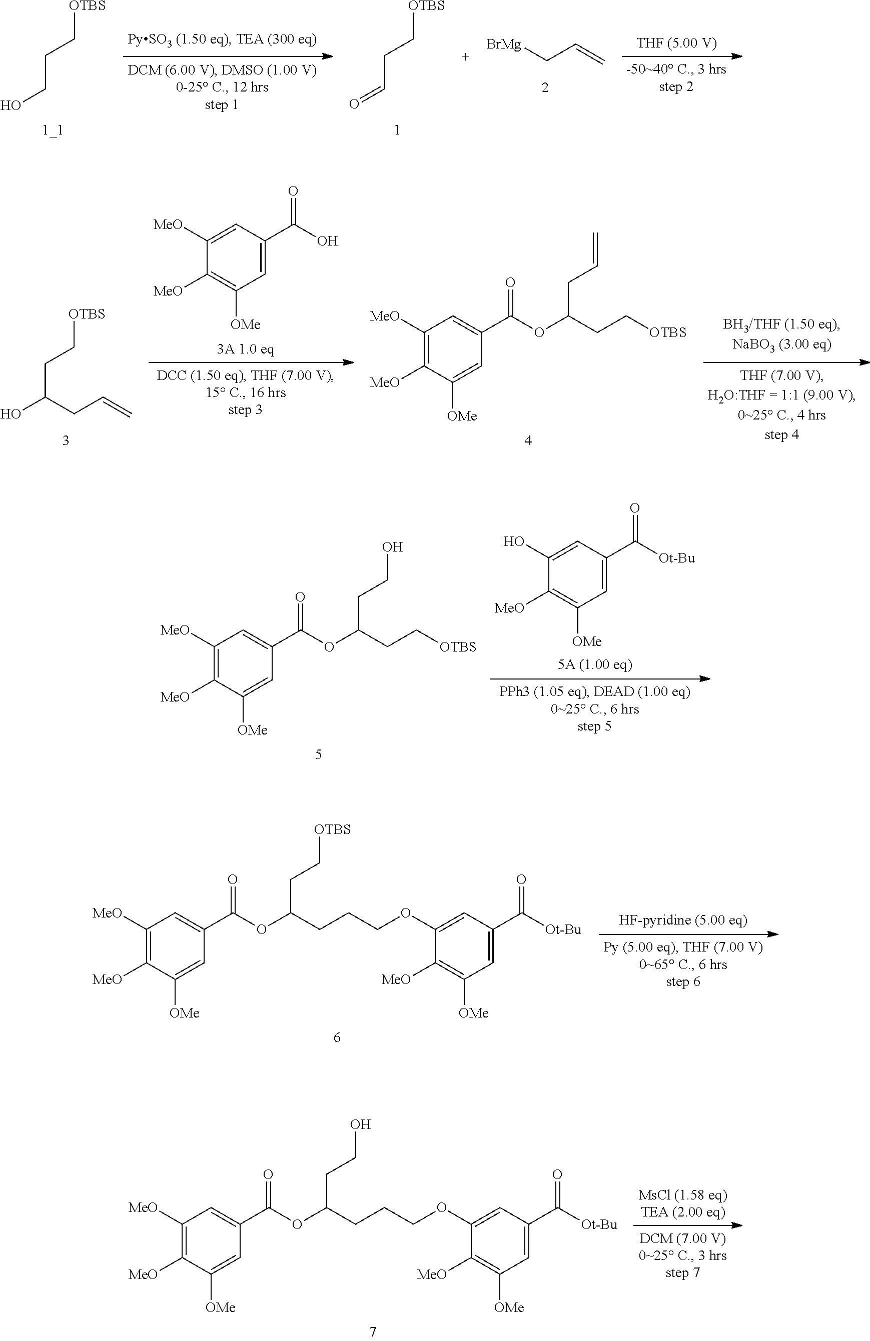

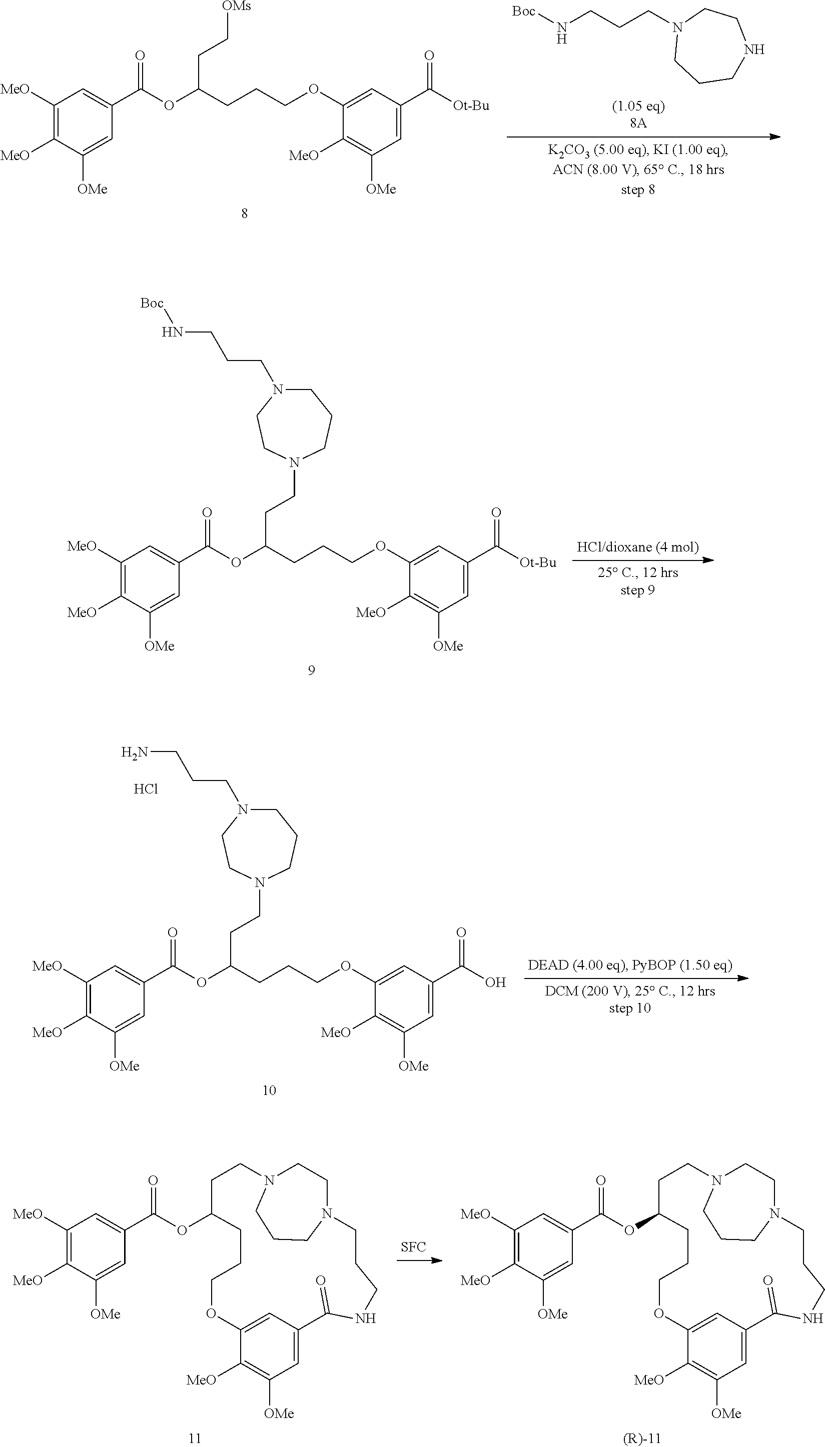

### Step 1:

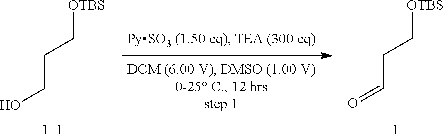 [SMILES: CC(C)(C)[Si](C)(C)OCCCO ; CC(C)(C)[Si](C)(C)OCCC=O]

At 0-5° C., Compound 1_1 (3.50 kg, 18.41 mol, 1.00 eq), DCM (21.0 L) and DMSO (3.50 L) were charged into the reactor. Then TEA (5.58 kg, 55.1 mol, 3.00 eq), Py·SO₃ (4.39 kg, 27.5 mol, 1.50 eq) into the mixture at 0-20° C. The reaction mixture was stirred at 20-25° C. for 12 hrs. An aqueous solution of 0.5 M citric acid (20.0 L) was slowly added into the mixture at 0˜20° C. and stirred for 10 min. The organic phase was separated, washed with an aqueous solution of 10% NaHCO₃ (20.0 L) and brine (20.0 L), dried over Na₂SO₄, filtered and concentrated to give the compound 1 (3.60 kg, crude) as brown oil.

Purity determined by quantitative NMR: 66.8%

1H NMR (400 MHZ, CDCl3-d) δ 9.79 (d, J=2.0 Hz, 1H), 3.97 (t, J=2.0 Hz, 2H), 2.57 (t, J=6.0 Hz, 2H), 0.89 (s, 9H), 0.05 (s, 6H)

### Step 2:

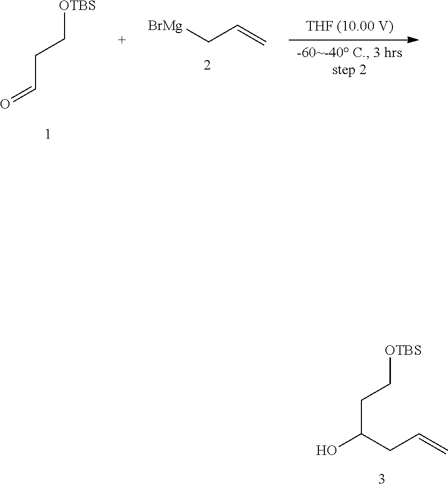

At 20° C., THF (28.8 L), Compound 2 (1 M, 22.9 L, 1.20 eq) was charged into the reactor then cooled −60-−50° C. Compound 1 (3.60 kg, 19.12 mol, 1.00 eq) in THF (7.20 L) was added into the mixture at −60-−50° C. The reaction mixture was stirred at −50-−40° C. for 3 hrs then slowly warmed to 0-10° C. The reaction was quenched by addition of an aqueous solution of 0.5 N HCl (20.0 L) between 0-10° C. The organic phase was separated, washed with brine (20.0 L), dried over Na₂SO₄, filtered and concentrated to give a brown oil. The oil was purified by column chromatography (SiO₂, n-hexane/ethyl acetate=I/O to 50/1) to give the compound 3 (2.10 kg, 9.11 mol, 48.0% yield) as yellow oil.

Note: Changed the charging sequence by adding the aldehyde to Grignard reagent, the yield increases from 40% to 48%.

1H NMR (400 MHZ, CDCl3-d) δ 5.72-5.94 (m, 1H), 4.97-5.18 (m, 2H), 3.75-3.94 (m, 3H), 3.37 (d, J=6.4 Hz, 1H), 2.17-2.31 (m, 2H), 1.60-1.71 (m, 2H), 0.88 (s, 9H), 0.03 (s, 6H).

### Step 3:

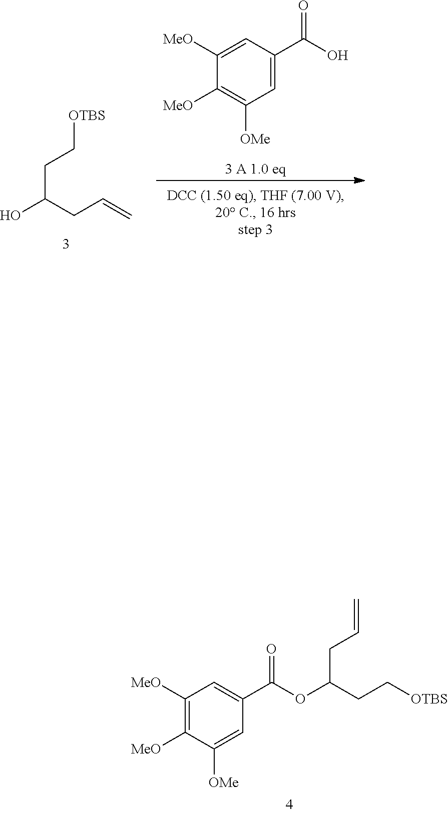 [SMILES: C=CCC(O)CCO[Si](C)(C)C(C)(C)C ; COc1cc(C(=O)O)cc(OC)c1OC ; C=CCC(CCO[Si](C)(C)C(C)(C)C)OC(=O)c1cc(OC)c(OC)c(OC)c1]

At 20-25° C., THF (14.70 L), Compound 3 (2.10 kg, 9.11 mol, 1.00 eq), Compound 3 A (1.93 kg, 9.11 mol, 1.00 eq), DCC (2.82 kg, 13.69 mol, 487 mL, 1.50 eq), DMAP (1.67 kg, 13.69 mol, 1.50 eq) were charged into the reactor. The reaction mixture was stirred at 20˜25° C. for 16 hrs. The reaction mixture was filtered and the filtrate concentrated under reduced pressure to give the residue. The residue was purified by column chromatography (n-hexane/ethyl acetate=100/0 to 90/10) to give the compound 4 (2.70 kg, 6.36 mol, 70% yield, 95.1% purity) as yellow oil.

1H NMR (400 MHZ, CDCl3-d) δ 7.28 (s, 2H), 5.74-5.92 (m, 1H), 5.22-5.33 (m, 1H), 5.02-5.17 (m, 2H), 3.90 (s, 9H), 3.67-3.76 (m, 2H), 2.41-2.57 (m, 2H), 1.87-2.00 (m, 2H), 0.89 (s, 9H), 0.05 (s, 6H)

### Step 4:

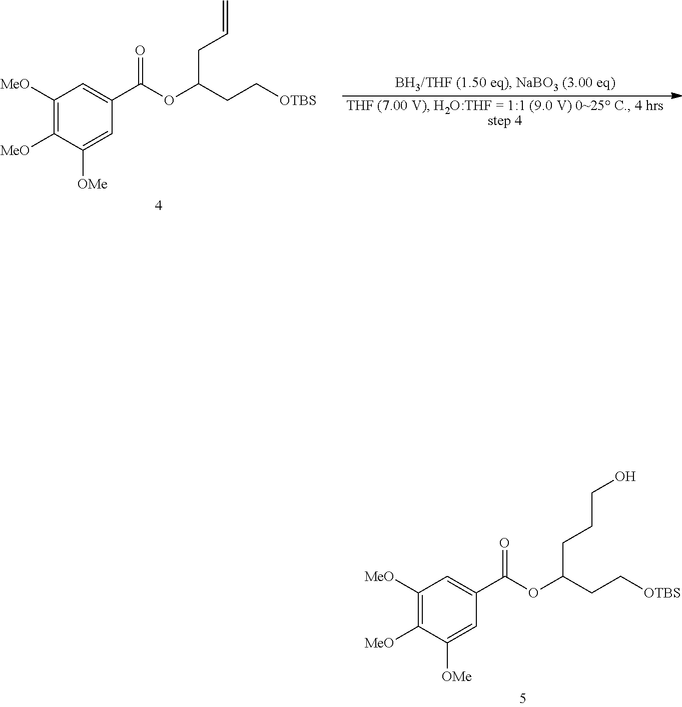

At 10-20° C., Compound 4 (2.40 kg, 5.66 mol, 1.00 eq) and THF (16.80 L) was charged into a 50.0 L reactor. BH₃·THF (1 M, 8.48 L, 1.50 eq) was added dropwise into the mixture at 0˜10° C. A mixture of H₂O (10.8 L) and THF (10.8 L) was added to quench the reaction between 0˜10° C. (Caution: evolution of H₂, and exothermal is observed.). NaBO3.4H₂O (2.61 kg, 16.9 mol, 3.00 eq) was charged by portions into the mixture at 0˜10° C., then the reaction mixture was stirred at 10˜25° C. for 4 hrs. The reaction was quenched by addition of an aqueous solution of 10% Na₂S₂O₃ (20.0 L) slowly at 0˜10° C. Ethyl acetate (7.50 L) was charged into the reactor at 10˜20° C. and stirred for 10 min. The organic phase was separated, washed with brine (5.00 L), dried over Na₂SO₄, filtered and concentrated to give the residue. The residue was purified by column chromatography (SiO₂, petroleum ether:ethyl acetate=10:1 to 1:1) to give the compound 5 (1.40 kg, 3.38 mol, 60% yield, 91.6% purity) as yellow oil.

1H NMR (400 MHZ, CDCl3-d) δ 7.26 (s, 2H), 5.16-5.33 (m, 1H), 3.88 (s, 9H), 3.55-3.78 (m, 4H), 1.56-2.01 (m, 6H), 0.87 (s, 9H), 0.02 (s, 6H).

### Step 5:

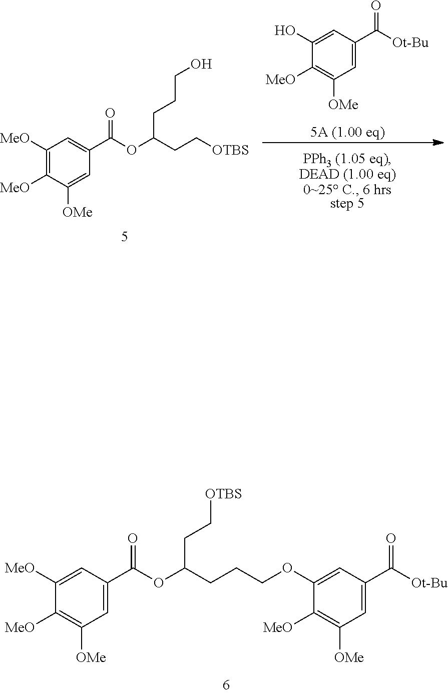

At 20° C., compound 5 (1.48 kg, 3.34 mol, 1.00 eq) and Tol (10.3 L), compound 5A (0.85 kg, 3.34 mol, 1.00 eq), PPh₃ (0.91 kg, 3.51 mol, 1.05 eq) were charged into the reactor. DEAD (0.58 kg, 3.34 mol, 1.00 eq) was added dropwise, (Exothermic phenomenon is observed during the addition process). After addition, the reaction mixture was stirred at 25° C. for 6 hrs, then the reaction mixture was stirred at −20° C. for 1 hr to precipitate part of OPPh₃. The reaction mixture was filtered and the filtrate concentrated under reduced pressure to give crude product. The crude product was purified by silica gel chromatography (n-hexane/Ethyl acetate=5/1) to give the compound 6 (1.38 kg, 2.03 mol, 81.2% purity) as colorless oil.

1H NMR (400 MHZ, CDCl3-d) δ 7.29 (s, 2H), 7.22 (d, J=1.6 Hz, 2H) 5.27-5.37 (m, 1H), 4.06 (s, 2H), 3.84-3.95 (m, 15H), 3.67-3.78 (m, 2H), 1.87-2.06 (m, 6H), 1.65 (s, 9H), 0.89 (s, 9H), 0.02 (s, 6H).

### Step 6:

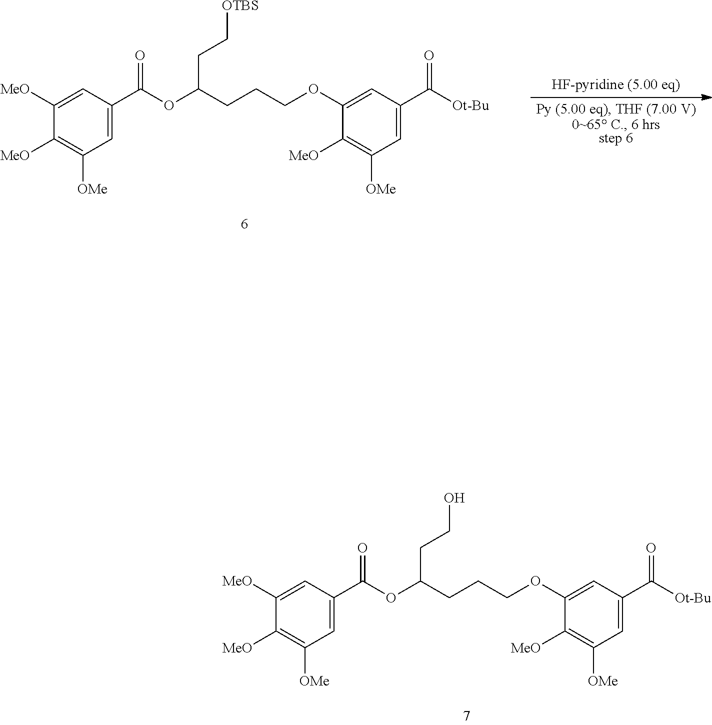

At 20° C., compound 6 (1.35 kg, 1.98 mol, 1.00 eq) and THF (9.45 L) were charged into the reactor. Pyridine (0.78 kg, 9.95 mol, 5.00 eq), HF-Pyridine (1.40 kg, 9.95 mol, 70% purity, 5.00 eq) were added to the reaction mixture at 0-10° C. The reaction mixture was stirred at 60˜65° C. for 6 hrs. An aqueous solution of 1 M citric acid (˜16.00 L) was added to the reaction mixture at 0˜20° C. and stirred for 10 min. The organic layer's pH was adjusted to pH ˜8 by addition of an aqueous solution of 10% NaHCO₃ (˜16.00 L). The organic layer was washed with brine (16.0 L), dried over Na₂SO₄, filtered and concentrated to give the compound 7 (1.09 kg, 81.0% purity) as yellow oil.

1H NMR (400 MHZ, CDCl3-d) δ 7.30 (s, 2H), 7.21-7.25 (m, 2H), 5.33-5.45 (m, 1H), 4.05-4.12 (m, 2H), 3.86-3.94 (m, 15H), 3.58-3.77 (m, 2H), 1.88-2.04 (m, 6H), 1.58 (s, 9H)

### Step 7:

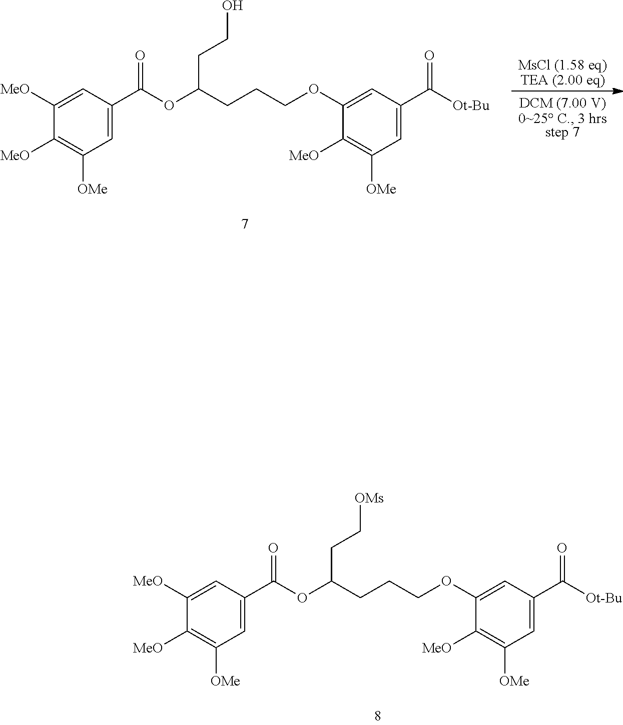

At 0° C., compound 7 (1.03 kg, 1.82 mol, 1.00 eq) and DCM (7.21 L) was charged into a 20.0 L reactor, then TEA (0.37 kg, 3.64 mol, 2.00 eq) was added. MsCl (0.33 kg, 2.88 mol, 1.58 eq) was added dropwise the reaction mixture at 0-5° C. The reaction mixture was stirred at 15˜25° C. for 3 hrs. An aqueous solution of 1 M citric acid (6.00 L) was slowly added to quench the reaction at 0˜20° C. and stirred for 10 min. The aqueous phase was separated. The organic layer was adjusted to pH=8 with an aqueous solution of 10% NaHCO₃ (6.00 L). The organic phase was separated, washed with brine (6.00 L), dried over Na₂SO₄, filtered and concentrated to give the compound 8 (1.14 kg, 1.77 mol, crude, 82% purity) as brown oil.

Purity determined by quantitative NMR: 87.3%

1H NMR (400 MHZ, CDCl3-d) δ 7.28 (s, 2H), 7.19-7.25 (m, 2H), 5.3-5.43 (m, 1H), 4.26-4.40 (m, 2H), 4.04-4.12 (m, 2H), 3.84-3.93 (m, 15H), 2.98 (s, 3H), 2.21 (q, J=6.0 Hz, 2H), 1.87-2.00 (m, 4H), 1.58 (s, 9H)

### Step 8:

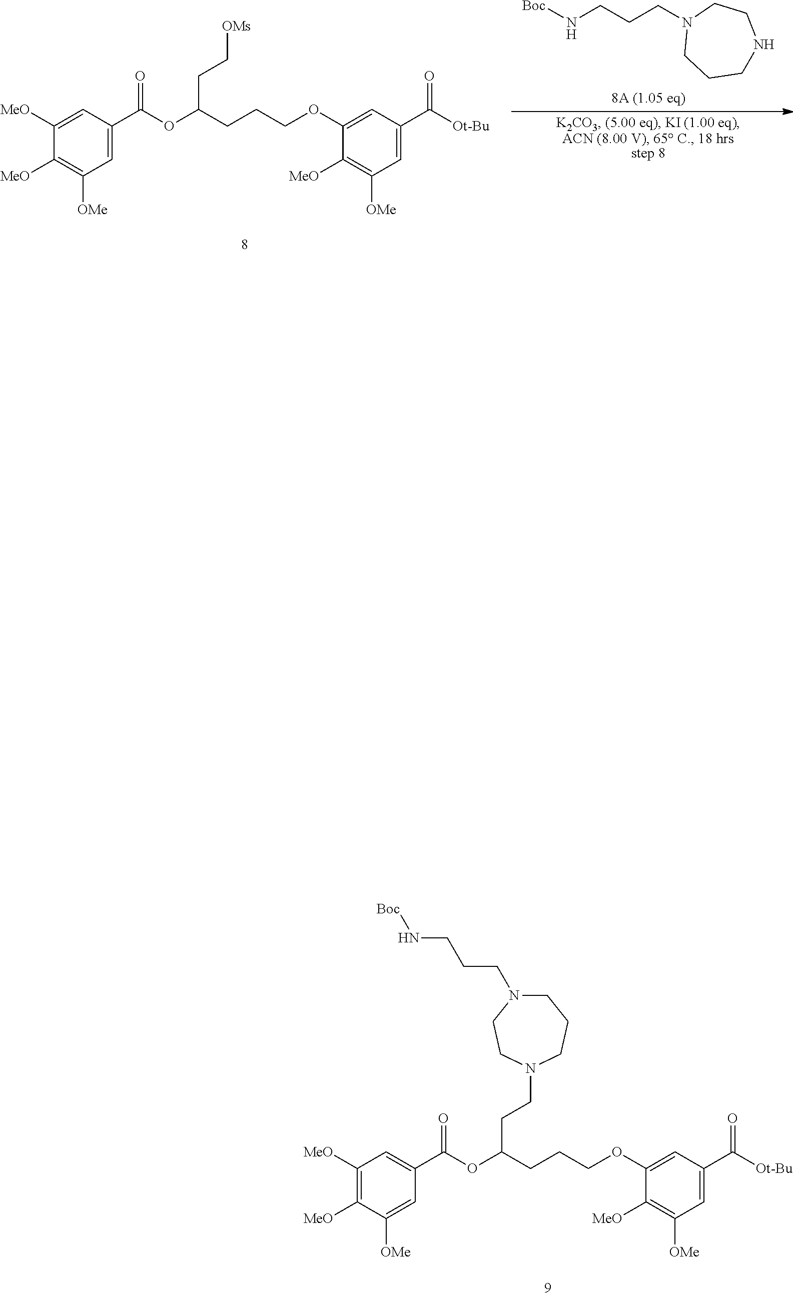 [SMILES: COc1cc(C(=O)OC(CCCOc2cc(C(=O)OC(C)(C)C)cc(OC)c2OC)CCOS(C)(=O)=O)cc(OC)c1OC ; CC(C)(C)OC(=O)NCCCN1CCCNCC1 ; "8A (1.05 eq)" ; "K2CO3, (5.00 eq), KI (1.00 eq),ACN (8.00 V), 65° C., 18 hrsstep 8" ; COc1cc(C(=O)OC(CCCOc2cc(C(=O)OC(C)(C)C)cc(OC)c2OC)CCN2CCCN(CCCNC(=O)OC(C)(C)C)CC2)cc(OC)c1OC]

At 25° C., compound 8 (1190 g, 1.85 mol, 1.00 eq) and ACN (9.52 L) was charged into a 20.0 L reactor. Then, compound 8A (548 g, 2.13 mol, 1.05 eq), K₂CO₃ (1279 g, 9.26 mol, 5.00 eq) and KI (307 g, 1.85 mol, 1.00 eq) were added. The reaction mixture was stirred at 65° C. for 18 hrs. The solvent was removed under reduced pressure to give the residue. H₂O (3.00 L) was added to the residue and extracted with EtOAc (3.00 L×3). The organic phase was separated, washed with brine (3.00 L), dried over Na₂SO₄, filtered and concentrated to give a residue. The residue was purified by column chromatography (SiO₂, Dichloromethane: Methanol=50/1 to 5/1) to give the compound 9 (1116 g, 1.39 mol, 75.0% yield) as yellow oil.

Purity determined by quantitative NMR: 91.8%

1H NMR (400 MHZ, CDCl3-d) δ 7.30 (s, 2H), 7.21 (s, 2H), 5.23-5.36 (m, 1H), 4.04-4.17 (m, 2H), 3.73-3.94 (m, 15H), 3.06 (t, J=6.8 Hz, 2H), 2.65-2.80 (m, 8H), 2.60 (t, J=7.6 Hz, 2H), 2.49 (t, J=7.6 Hz, 2H), 1.86-2.03 (m, 6H), 1.76-1.85 (m, 2H), 1.61-1.68 (m, 2H), 1.58 (s, 9H), 1.43 (s, 9H).

### Step 9:

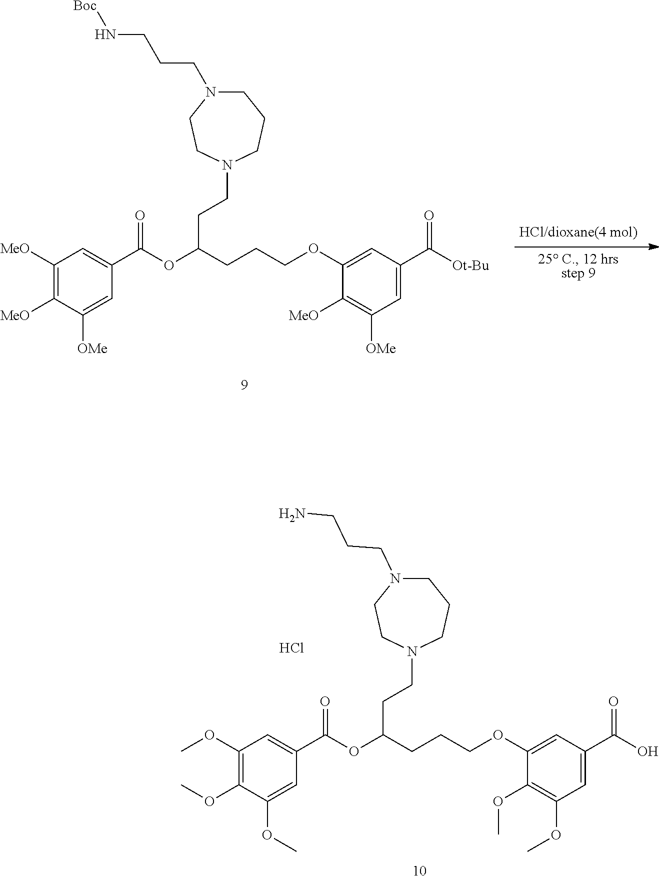 [SMILES: COc1cc(C(=O)OC(CCCOc2cc(C(=O)OC(C)(C)C)cc(OC)c2OC)CCN2CCCN(CCCNC(=O)OC(C)(C)C)CC2)cc(OC)c1OC ; "25° C., 12 hrsstep 9" ; "HCl/dioxane(4 mol)" ; COc1cc(C(=O)OC(CCCOc2cc(C(=O)O)cc(OC)c2OC)CCN2CCCN(CCCN)CC2)cc(OC)c1OC ; Cl]

At 0-5° C., a solution of HCl in dioxane (4 mol, 7.60 L) and compound 9 (1086 g, 1.35 mol, 1.00 eq) was charged into a 20.0 L reactor. The reaction mixture was stirred at 25° C. for 12 hrs. The solvent was removed under reduced pressure to give the compound 10 (1050 g, as HCl Salt) as yellow solid.

Purity determined by quantitative NMR: 75.2%

1H NMR (400 MHZ, MeOD-d4) δ 7.29 (s, 2H), 7.26 (s, 2H) 5.2-5.37 (m, 1H), 4.11 (s, 2H), 3.94 (brs, 4H), 3.78-3.90 (m, 15H), 3.33-3.45 (m, 4H), 3.08 (t, J=7.6 Hz, 2H), 2.12-2.49 (m, 6H), 1.90-2.09 (m, 4H)

### Step 10:

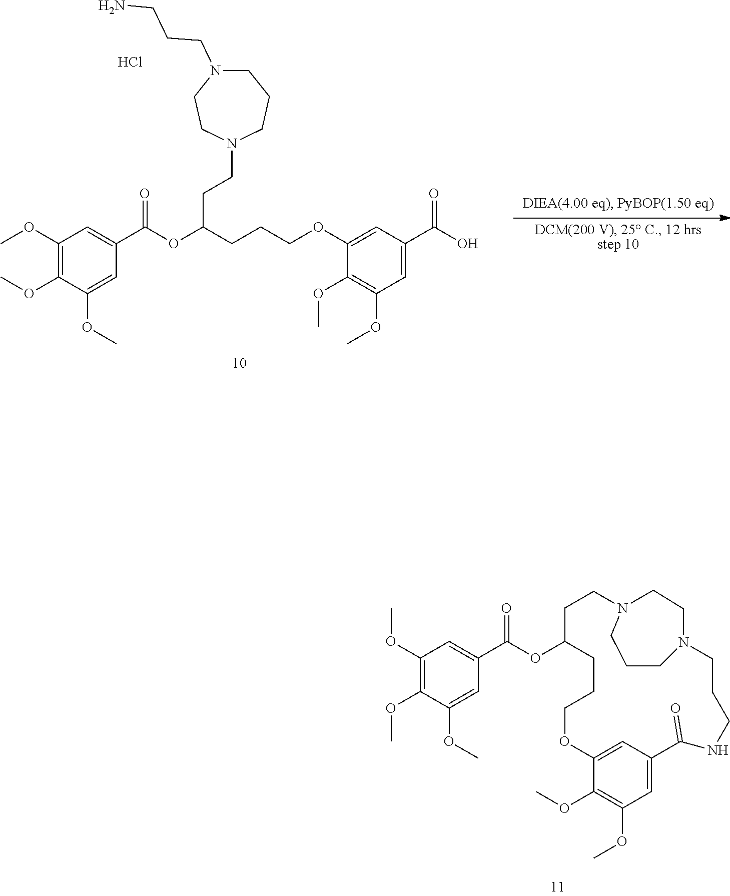 [SMILES: COc1cc(C(=O)OC(CCCOc2cc(C(=O)O)cc(OC)c2OC)CCN2CCCN(CCCN)CC2)cc(OC)c1OC ; Cl ; "DCM(200 V), 25° C., 12 hrsstep 10" ; "DIEA(4.00 eq), PyBOP(1.50 eq)" ; COc1cc(C(=O)OC2CCCOc3cc(cc(OC)c3OC)C(=O)NCCCN3CCCN(CC2)CC3)cc(OC)c1OC]

At 20° C., compound 10 (1050 g, 1.53 mol, 1.00 eq, HCl) and DCM (210 L) were charged into the reactor. Then, DIEA (793 g, 6.13 mol, 4.00 eq) and PyBOP (38.4 g, 2.29 mol, 1.50 eq) were added to the reactor at 20° C. The reaction mixture was stirred at 25° C. for 12 hrs. The reaction mixture was concentrated at 35-40° C. to give the residue. The residue was triturated with MeOH (4.2 L, 4.00 V) at 20° C. for 60 min. The mixture was filtered and the cake collected to give the compound 11 (470 g, 34.94 mmol, 48.6% yield) as white solid.

Purity determined by quantitative NMR: 75.2%

1H NMR (400 MHZ, MeOD-d4) δ 7.31 (s, 2H), 7.20 (d, J=1.8 Hz, 1H), 7.13 (d, J=1.8 Hz, 1H), 5.49 (s, 1H), 4.31 (br d, J=8.3 Hz, 1H), 4.18 (br s, 1H), 3.85-3.89 (m, 9H), 3.81 (d, J=7.3 Hz, 6H), 3.56-3.66 (m, 1H), 3.38-3.49 (m, 1H), 2.97 (td, J=3.2, 10.3 Hz, 1H), 2.84-2.91 (m, 2H), 2.74-2.84 (m, 3H), 2.61-2.73 (m, 4H), 2.56 (br t, J=6.5 Hz, 2H), 1.86-1.95 (m, 5H), 1.73-1.85 (m, 5H).

### SFC-Chiral Separation of Compound 11:

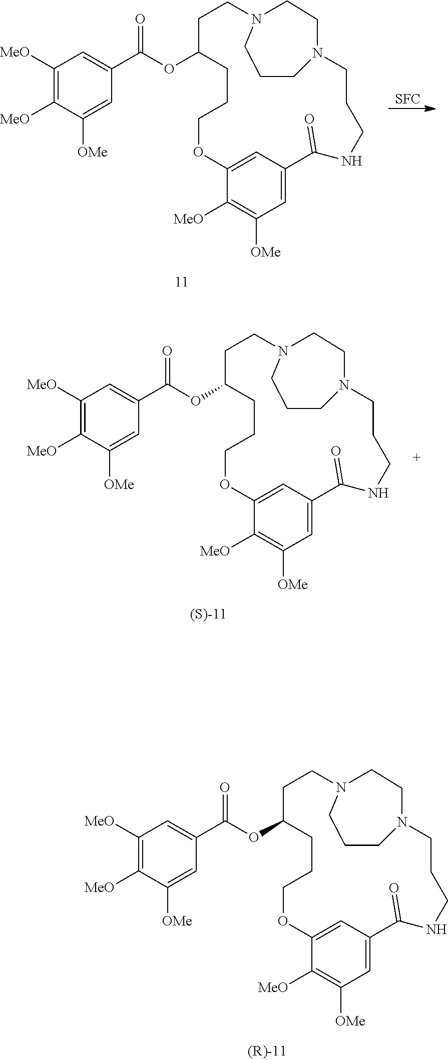

Enantiomers of the racemic compound 11 (470 g) were separated by chiral-SFC (Supercritical Fluid Chromatography-Column: Phenomenex-Cellulose-2 (250 mm*30 mm, 10 μm); mobile phase: [0.1% NH₃H₂O MEOH]; B %: 60%-60%, 10 min) to give the compound(S)-11 (170 g) and compound (R)-11 (165 g) as white solids.

### Compound(S)-11:

LCMS (method 1) (ESI position ion) m/z: 630.2 (M+H)+ (calculated: 630.3), purity >99%

Chiral HPLC (method 1): retention time=3.836 min, ee>99%

1H NMR (400 MHZ, MeOD-d4) δ 7.31 (s, 2H), 7.20 (d, J=1.8 Hz, 1H), 7.13 (d, J=1.8 Hz, 1H), 5.49 (s, 1H), 4.31 (br d, J=8.3 Hz, 1H), 4.18 (br s, 1H), 3.85-3.89 (m, 9H), 3.81 (d, J=7.3 Hz, 6H), 3.56-3.66 (m, 1H), 3.38-3.49 (m, 1H), 2.97 (td, J=3.2, 10.3 Hz, 1H), 2.84-2.91 (m, 2H), 2.74-2.84 (m, 3H), 2.61-2.73 (m, 4H), 2.56 (br t, J=6.5 Hz, 2H), 1.86-1.95 (m, 5H), 1.73-1.85 (m, 5H).

### Compound (R)-11:

LCMS (method 1) (ESI position ion) m/z: 630.2 (M+H)+ (calculated: 630.3), purity >99%

Chiral SFC (method 1): retention time=6,560 min, ee>99%

1H NMR (400 MHZ, MeOD-d4) δ 7.31 (s, 2H), 7.20 (d, J=1.8 Hz, 1H), 7.13 (d, J=1.8 Hz, 1H), 5.49 (s, 1H), 4.31 (br d, J=8.3 Hz, 1H), 4.18 (br s, 1H), 3.85-3.89 (m, 9H), 3.81 (d, J=7.3 Hz, 6H), 3.56-3.66 (m, 1H), 3.38-3.49 (m, 1H), 2.97 (td, J=3.2, 10.3 Hz, 1H), 2.84-2.91 (m, 2H), 2.74-2.84 (m, 3H), 2.61-2.73 (m, 4H), 2.56 (br t, J=6.5 Hz, 2H), 1.86-1.95 (m, 5H), 1.73-1.85 (m, 5H).

Step 1 of the Conversion of(S)-11 to (R)-11:

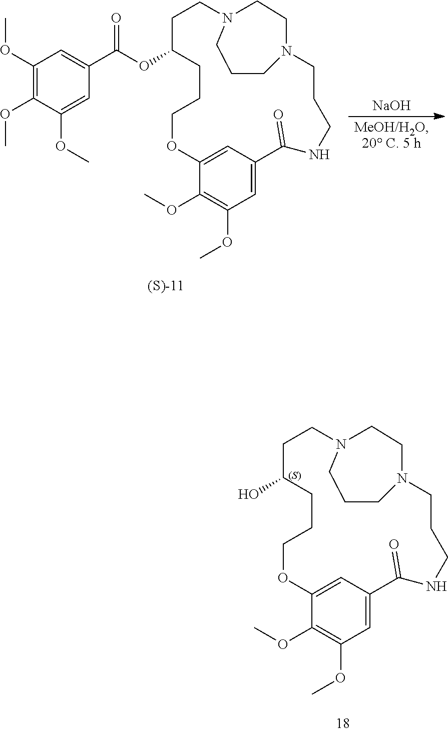

To a solution of compound(S)-11 (150 g, 238.19 mmol, 1 eq) in MeOH (800 mL) and H₂O (400 mL) was added NaOH (28.58 g, 714.58 mmol, 3 eq). The mixture was stirred at 20° C. for 5 hr. The solvent MeOH was removed under reduced pressure at 25° C. The mixture was diluted with H₂O (1500 mL) and extracted with DCM (500 mL×3). The organic layer was washed with brine, dried by Na₂SO₄. The solution was concentrated to afford compound 18 (116.5 g, crude) as yellow solid.

LCMS (method 1) (ESI position ion) m/z: 436.2 (M+H)+ (calculated: 436.3)

1H NMR (400 MHZ, MeOD-d4) δ 7.15 (dd, J=1.8, 7.6 Hz, 2H), 4.30-4.13 (m, 2H), 3.99-3.90 (m, 1H), 3.88 (s, 3H), 3.83 (s, 3H), 3.57-3.40 (m, 2H), 2.88-2.52 (m, 12H), 2.05-1.92 (m, 1H), 1.91-1.71 (m, 5H), 1.71-1.51 (m, 4H).

Step 2 of the Conversion of(S)-11 to (R)-11:

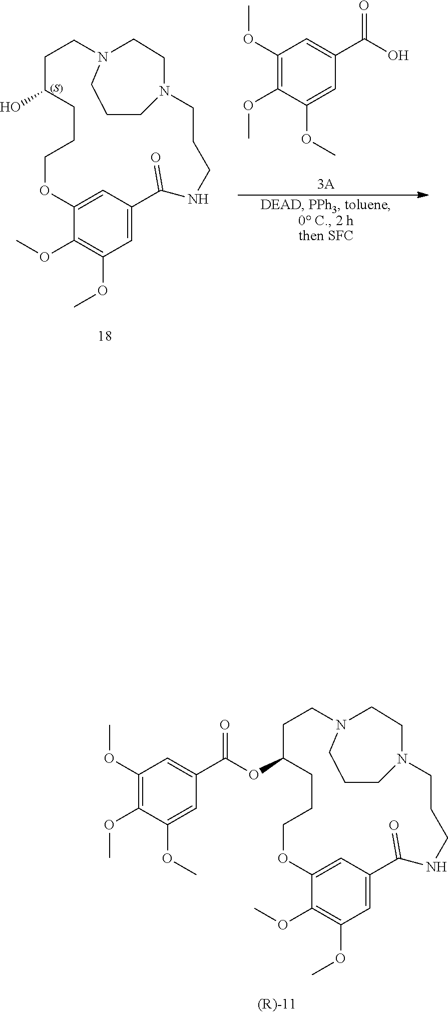

A mixture of compound 18 (10.00 g, 22.96 mmol, 1 eq), compound 3A (14.62 g, 68.88 mmol, 3 eq) and PPh₃ (30.11 g, 114.80 mmol, 5 eq) in toluene (250 mL) was added DEAD (19.99 g, 114.80 mmol, 20.87 mL, 5 eq) dropwise at 0° C. The mixture was stirred at 0° C. for 2 hr under nitrogen atmosphere. The reaction mixture was filtered by column chromatography (SiO₂, Petroleum ether/Ethyl acetate=1/0 to 0/1 and DCM/MeOH=10/1 to 1/1) to give the crude product. The crude product was purified by prep-HPLC (column: Welch Xtimate C18 250*50 mm*10 μm; mobile phase: [water (FA)-ACN]; B %: 2%-32%, 15 min). The purified solution was concentrated and adjusted pH with NaHCO₃ to 7-8 at 0° C. The solution was extracted with DCM (500 mL×2). The organic layer was washed with brine, dried over Na₂SO₄. The solution was concentrated to afford compound (R)-11 (4.7 g, 33% yield) as a white solid. This reaction was carried out in 12 batches and totally affording 50 g of (R)-11 with a ee=60%. The compound was further purified by chiral SFC in the condition below to afford the compound (R)-11 (35.5 g) as a white solid.

LCMS (method 1) (ESI position ion) m/z: 630.2 (M+H)+ (calculated: 630.3), purity >99%

Chiral SFC (method 1): retention time=6,560 min, ee>99%

1H NMR (400 MHZ, MeOD-d4) δ 7.31 (s, 2H), 7.20 (d, J=1.8 Hz, 1H), 7.13 (d, J=1.8 Hz, 1H), 5.49 (s, 1H), 4.31 (br d, J=8.3 Hz, 1H), 4.18 (br s, 1H), 3.85-3.89 (m, 9H), 3.81 (d, J=7.3 Hz, 6H), 3.56-3.66 (m, 1H), 3.38-3.49 (m, 1H), 2.97 (td, J=3.2, 10.3 Hz, 1H), 2.84-2.91 (m, 2H), 2.74-2.84 (m, 3H), 2.61-2.73 (m, 4H), 2.56 (br t, J=6.5 Hz, 2H), 1.86-1.95 (m, 5H), 1.73-1.85 (m, 5H). 5H), 1.83-1.72 (m, 5H).

Column: Chiralpak AD-3 50×4.6 mm I.D., 3 μm

Mobile phase: Phase A for CO2, and Phase B for IPA (0.05% DEA);

Gradient elution: B in A from 5% to 40%

Flow rate: 3 mL/min; Detector: PDA

Column Temp: 35 C; Back Pressure: 100 Bar

SFC: tR=9.658 min, 100% e.e value

### Example 2. Synthesis of Compound (R)-11

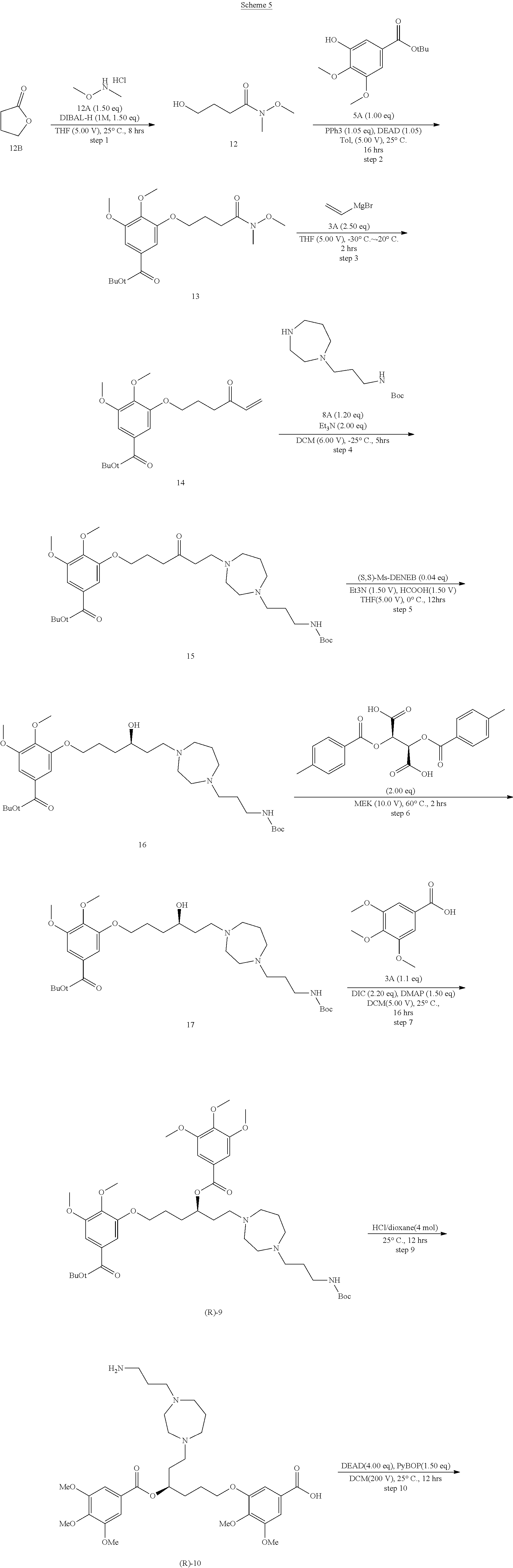

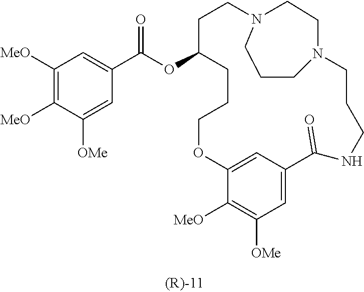 [SMILES: COc1cc(C(=O)O[C@@H]2CCCOc3cc(cc(OC)c3OC)C(=O)NCCCN3CCCN(CC2)CC3)cc(OC)c1OC]

### Step 1:

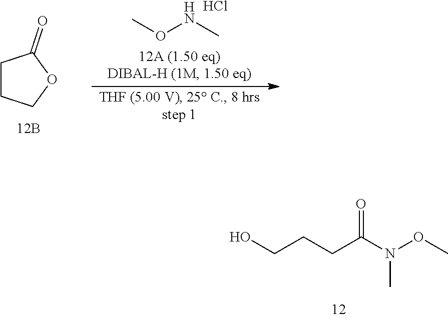

At 15-25° C., compound 12A (210 g, 1.50 eq, 1.57 mol) was dissolved in THF (450 mL, 5.00 V). To the reaction mixture was added DIBAL-H (1.57 L, 1.50 eq, 1.57 mol) dropwise at 0-10° C. The reaction mixture was stirred at 15-25° C. for 2 hrs. The compound 12B (90.0, 1.00 eq, 1.05 mol) was added to the reaction mixture at 0-10° C. dropwise. The reaction mixture was stirred at 15-25° C. for 5 hrs. H₂O (63.0 mL) was added at 0-10° C. dropwise, then an aqueous solution of NaOH (15%, 63.0 mL) slowly, then additional H₂O (157 mL) at 0-10° C. slowly. The reaction mixture was stirred at 15-25° C. for 15 mins then dried over MgSO₄. The solvent was removed under reduced pressure to give the compound 12 (70.0 g, 0.48 mol, 45.5% yield) as yellow oil.

### Step 2:

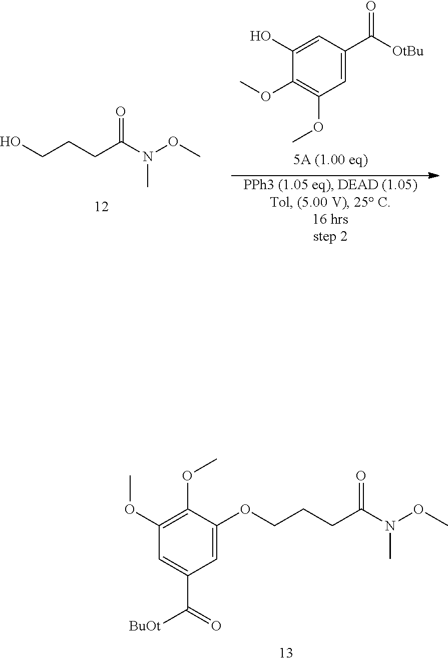

At 15-25° C., compound 12 (30.0 g, 1.00 eq), compound 5A (51.8 g, 1.00 eq), PPh₃ (56.1 g, 1.05 eq) and toluene (150 mL, 5.00 V) were charged into the reactor. DEAD (37.2 g, 1.05 eq) was added dropwise to the reaction mixture at 0-10° C. The reaction mixture was stirred at 15-25° C. for 24 hrs. The solvent was removed under reduced pressure and the residue was purified by silica column chromatography (SiO₂, Petroleum ether/Ethyl acetate=100/1 to 0/1) to give the compound 13 (50.0 g, 0.13 mol) as yellow solid.

1H NMR (400 MHZ, CDCl3-d) δ 7.24 (s, 2H) 4.08-4.16 (m, 2H) 3.84-3.94 (m, 6H) 3.62-3.76 (m, 3H) 3.13-3.26 (m, 3H) 2.59-2.74 (m, 2H) 2.12-2.32 (m, 2H) 1.52-1.63 (m, 9H)

### Step 3:

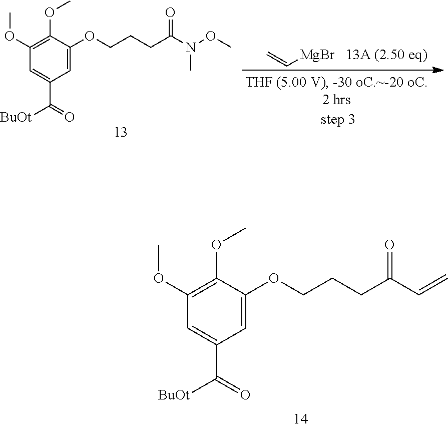

At 15-25° C., compound 13 (45.0 g, 1.00 eq) was dissolved in THF (225 mL, 5.00 V). At −30° C., the compound 13A (293 mL, 1.00 eq, 1M) was added to the reaction mixture dropwise. The reaction mixture was stirred at −30° C. for 2 hrs. HCl (1.35 L, 1M in H₂O, 30.0 V) was slowly added −30° C. Ethyl acetate (225 mL, 5.00 V) was added. The organic phase was separated, washed with brine, dried over Na₂SO₄ and concentrated under reduced pressure to give the crude product. The crude product was purified by silica column chromatography (SiO₂, Petroleum ether/Ethyl acetate=100/1 to 0/1) to give the compound 14 (27.0 g, 75.5 mmol, 64.3% yield, 98.3% purity) as yellow oil.

1H NMR (400 MHZ, CDCl3-d) δ 7.21-7.25 (m, 2H) 6.20-6.53 (m, 2H) 5.80-5.89 (m, 1H) 4.03-4.14 (m, 2H) 3.81-3.92 (m, 6H) 2.77-2.94 (m, 2H) 2.06-2.25 (m, 2H) 1.46-1.67 (m, 10H)

### Step 4:

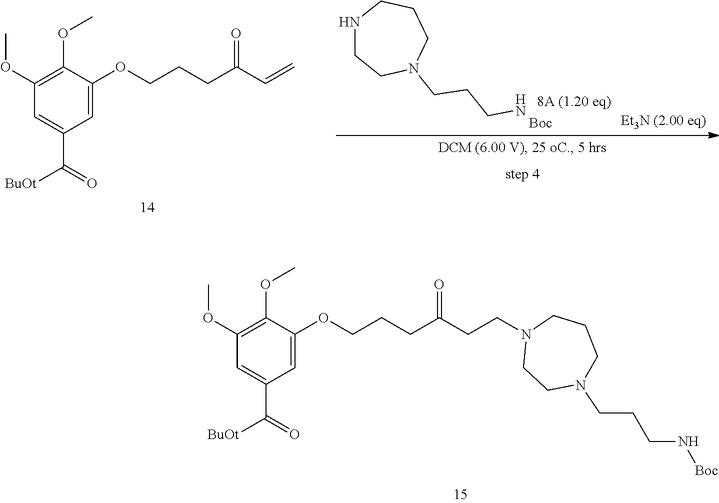

At 15-25° C., compound 14 (27.0 g, 1.00 eq) was dissolved in DCM (135 mL, 5.00 V). The compound 8A (24.7 g, 1.00 eq) and Et₃N (15.6 g, 2.00 eq) was added and the reaction mixture was stirred for 12 hrs. The reaction mixture was concentrated to give the crude compound 15 (42.0 g, 69.1 mmol) as a yellow oil.

1H NMR (400 MHz, MeOD-d4) δ 7.21-7.28 (m, 2H) 4.01-4.13 (m, 2H) 3.75-3.91 (m, 6H) 2.99-3.16 (m, 3H) 2.63-2.85 (m, 13H) 2.44-2.58 (m, 3H) 2.02-2.12 (m, 2H) 1.75-1.86 (m, 2H) 1.61-1.66 (m, 2H) 1.59 (s, 9H) 1.43 (s, 9H)

### Step 5:

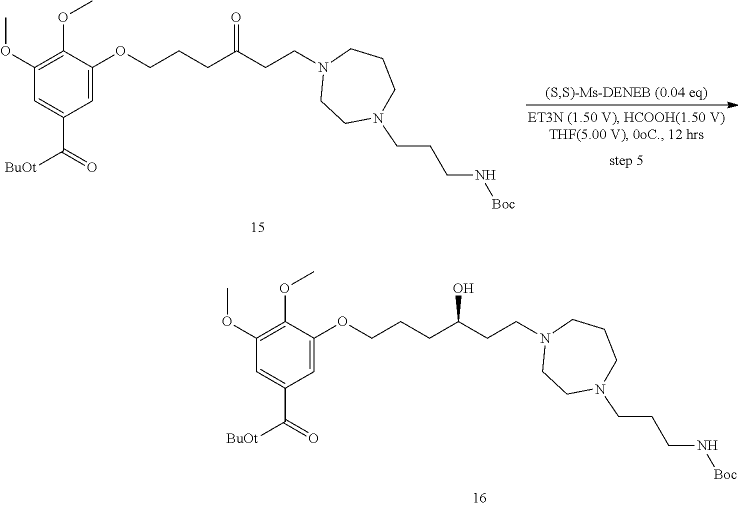

At 15-25° C., to a solution of compound 15 (3.00 g, 1.00 eq), HCOOH/Et₃N (9.00 mL, 1:1, 3.00 V) in THF (15.0 mL, 5.00 V) was added (S,S)-Ms-DENEB catalyst (0.11 g, 0.04 eq). The reaction mixture was stirred at 15-25° C. for 12 hrs. H₂O (9.00 mL, 3.00 V) and DCM (9.00 mL, 3.00 V) was added to the reaction mixture. The organic phase was separated, washed with brine, dried over Na₂SO₄ and concentrated under reduced pressure to give the crude product. The crude product was purified by silica column chromatography (SiO₂, Petroleum ether/Ethyl acetate=100/1 to 0/1) to give the compound 16 (1.80 g, 2.89 mmol, 98.1% purity) as yellow oil. LCMS (method 2) (ESI position ion) m/z: 630.2 (M+H)+ (calculated: 630.3)

Chiral HPLC (method 2): retention time=21.398 and 23.972 min, ee=85.9%

### Step 6:

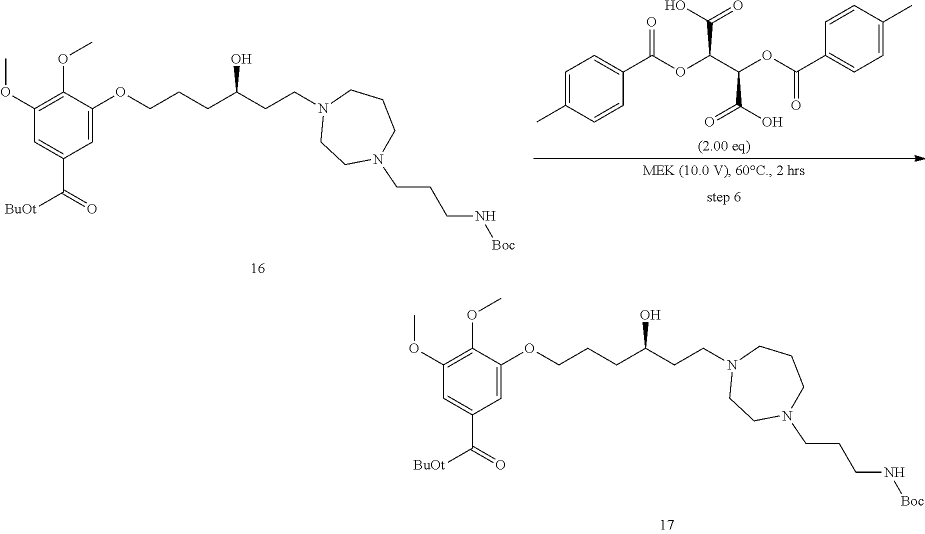

The compound 16 (1.00 eq) in butanone (MEK) (5.00 V) at 15-25° C. then the mixture was stirred at 55-60° C. for 2 hrs. Di-p-toluoyl-1-tartaric acid (2.00 eq) was added and the mixture stirred at 10-20° C. for 12 hrs.

The mixture was concentrated to give the crude product. (Reddish brown solid) The solid was triturated with butanone (10.0 V) at 25° C. for 30 mins. (white solid). The mixture was filtered and the filter cake was washed with butanone (1.00 V) for twice. The solid was dissolved in water (3.00 V). A saturated solution sodium carbonate was added into the mixture to adjust pH=11. DCM (3.00 V) was added into the mixture and the organic phase was separated, washed with brine, dried over Na₂SO₄, concentrated under reduced pressure to give the compound 17 as a reddish brown oil.

Chiral HPLC (method 2): retention time=18.335 and 20.673 min, ee=95.9%

### Step 7:

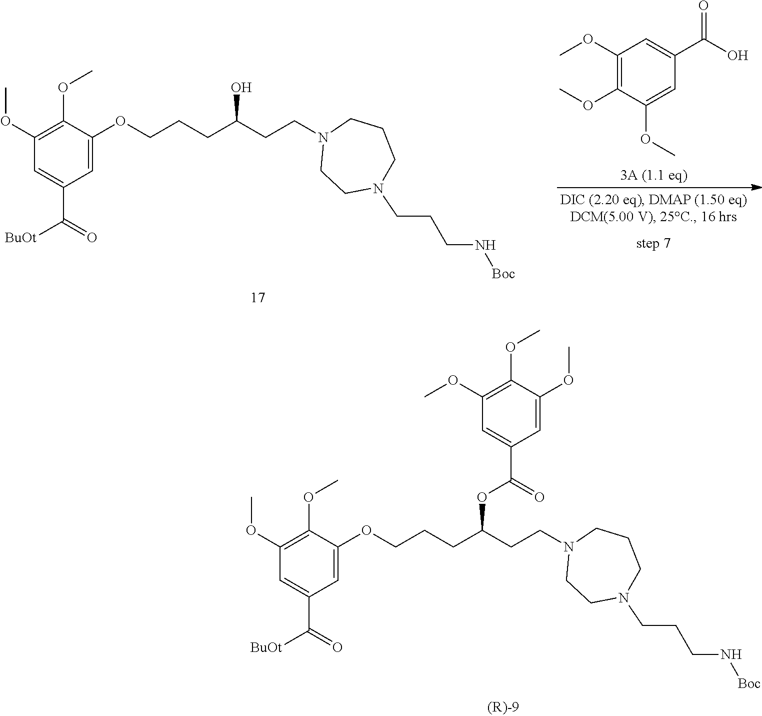

At 15-25° C., to a solution of compound 17 (1.00 g, 1.00 eq), compound 3A (1.20 eq) in DCM (5.00 V) was added DIC (2.20 eq) and DMAP (1.50 eq). The reaction mixture was stirred at 15-25° C. for 16 hrs. H₂O (3.00 V) and DCM (3.00 V) were added and the organic phase was separated, washed with brine, dried over Na₂SO₄ and concentrated under reduced pressure to give the crude product. The crude product was purified by silica column chromatography (SiO₂, Petroleum ether/Ethyl acetate=100/1 to 0/1) to give the compound (R)-9 (600 mg, 45% yield). LCMS (method 2) (ESI position ion) m/z: 804.4 (M+H)+ (calculated: 804.5)

### Step 8:

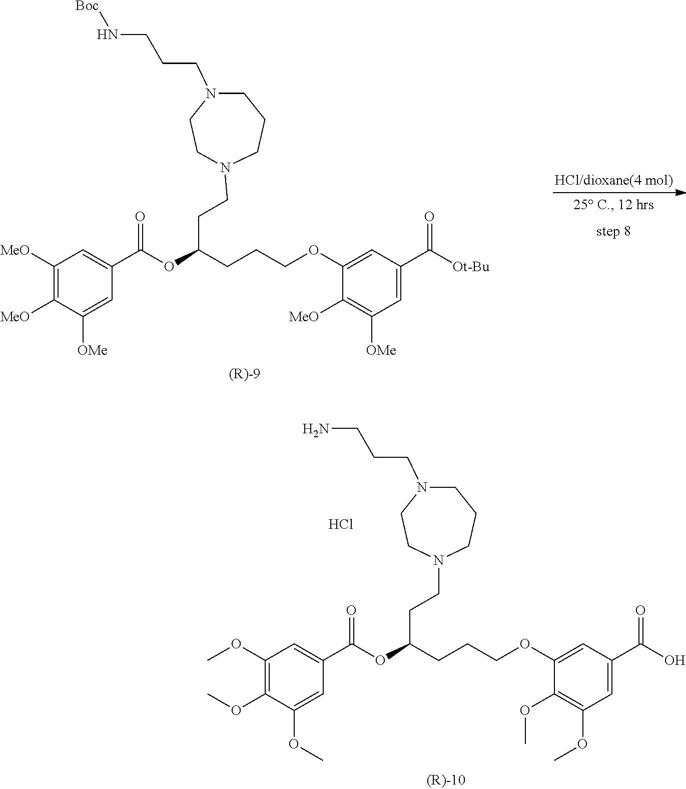 [SMILES: COc1cc(C(=O)O[C@H](CCCOc2cc(C(=O)OC(C)(C)C)cc(OC)c2OC)CCN2CCCN(CCCNC(=O)OC(C)(C)C)CC2)cc(OC)c1OC ; COc1cc(C(=O)O[C@H](CCCOc2cc(C(=O)O)cc(OC)c2OC)CCN2CCCN(CCCN)CC2)cc(OC)c1OC ; Cl]

At 0-5° C., a solution of HCl in dioxane (4 mol, 7.60 L) and compound (R)-9 (1086 g, 1.35 mol, 1.00 eq) was charged into a 20.0 L reactor. The reaction mixture was stirred at 25° C. for 12 hrs. The solvent was removed under reduced pressure to give the compound (R)-10 (1050 g, as HCl Salt) as yellow solid.

Purity determined by quantitative NMR: 75.2%

1H NMR (400 MHZ, MeOD-d4) δ 7.29 (s, 2H), 7.26 (s, 2H) 5.2-5.37 (m, 1H), 4.11 (s, 2H), 3.94 (brs, 4H), 3.78-3.90 (m, 15H), 3.33-3.45 (m, 4H), 3.08 (t, J=7.6 Hz, 2H), 2.12-2.49 (m, 6H), 1.90-2.09 (m, 4H)

### Step 9:

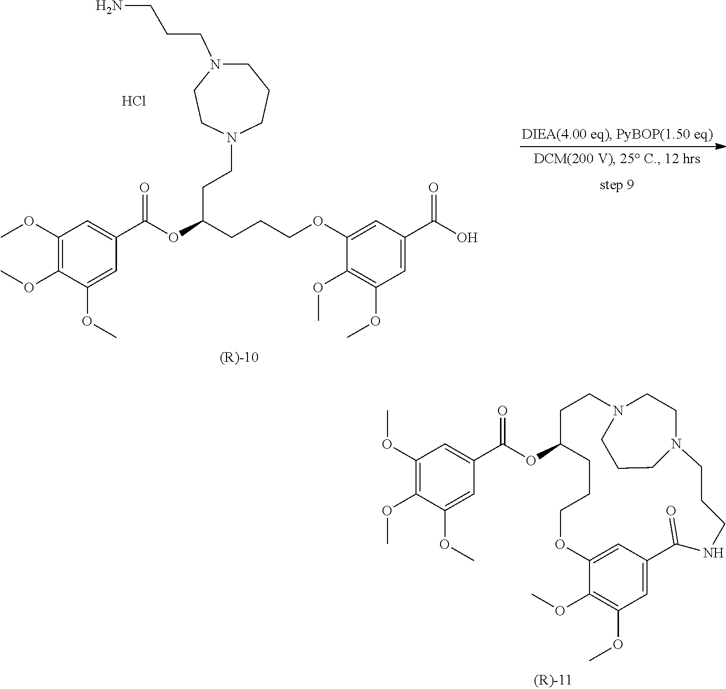 [SMILES: COc1cc(C(=O)O[C@H](CCCOc2cc(C(=O)O)cc(OC)c2OC)CCN2CCCN(CCCN)CC2)cc(OC)c1OC ; Cl ; COc1cc(C(=O)O[C@@H]2CCCOc3cc(cc(OC)c3OC)C(=O)NCCCN3CCCN(CC2)CC3)cc(OC)c1OC]

At 20° C., compound (R)-10 (1050 g, 1.53 mol, 1.00 eq, HCl) and DCM (210 L) were charged into the reactor. Then, DIEA (793 g, 6.13 mol, 4.00 eq) and PyBOP (38.4 g, 2.29 mol, 1.50 eq) were added to the reactor at 20° C. The reaction mixture was stirred at 25° C. for 12 hrs. The reaction mixture was concentrated at 35-40° C. to give the residue. The residue was triturated with MeOH (4.2 L, 4.00 V) at 20° C. for 60 min. The mixture was filtered and concentrated in vacuum to give the compound (R)-11 (470 g, 34.94 mmol, 48.6% yield) as white solid. Purity determined by quantitative NMR: 75.2%

1H NMR (400 MHZ, MeOD-d4) δ 7.31 (s, 2H), 7.20 (d, J=1.8 Hz, 1H), 7.13 (d, J=1.8 Hz, 1H), 5.49 (s, 1H), 4.31 (br d, J=8.3 Hz, 1H), 4.18 (br s, 1H), 3.85-3.89 (m, 9H), 3.81 (d, J=7.3 Hz, 6H), 3.56-3.66 (m, 1H), 3.38-3.49 (m, 1H), 2.97 (td, J=3.2, 10.3 Hz, 1H), 2.84-2.91 (m, 2H), 2.74-2.84 (m, 3H), 2.61-2.73 (m, 4H), 2.56 (br t, J=6.5 Hz, 2H), 1.86-1.95 (m, 5H), 1.73-1.85 (m, 5H).

### Example 3. Synthesis of Compound (R)-11

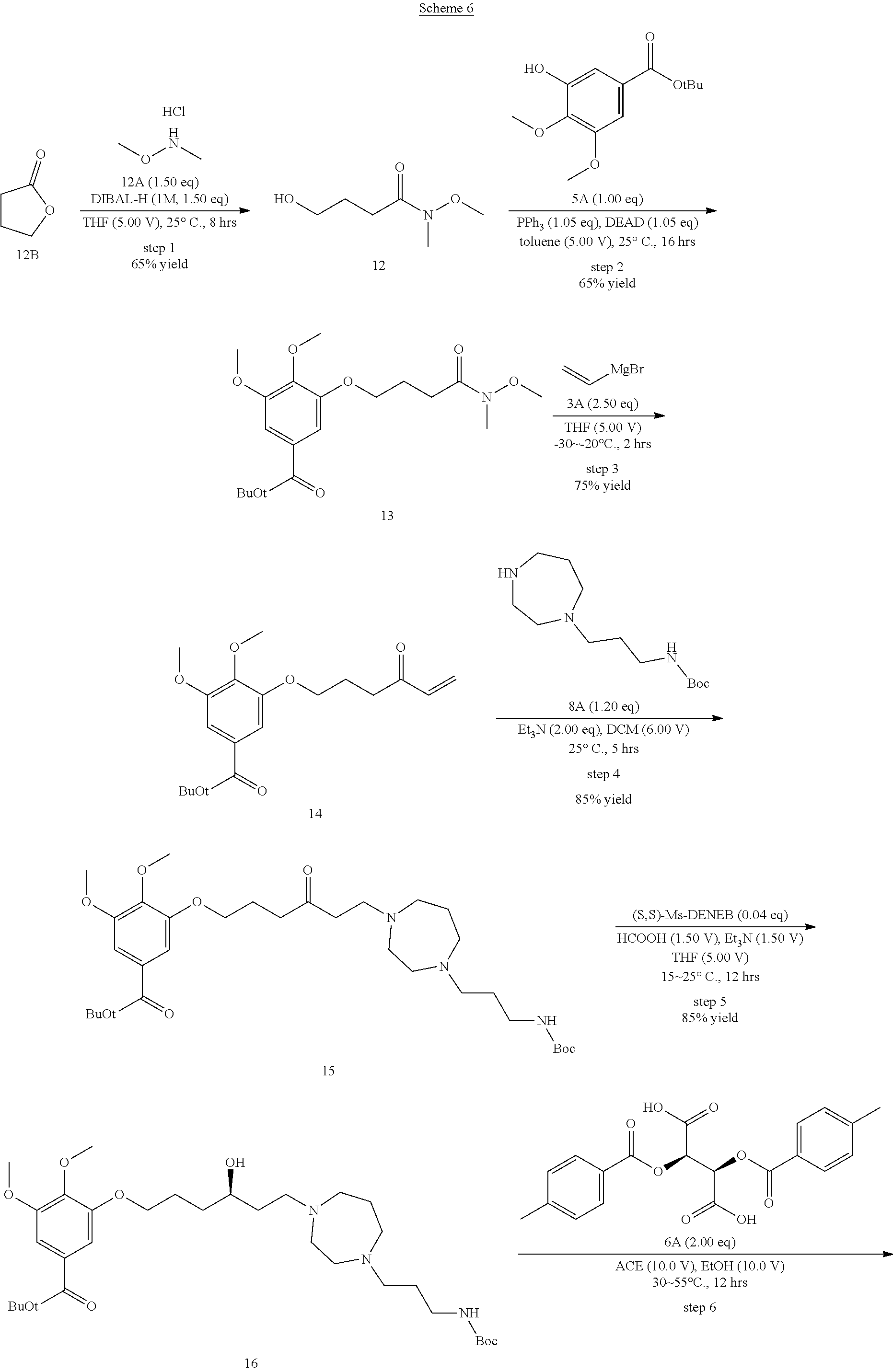

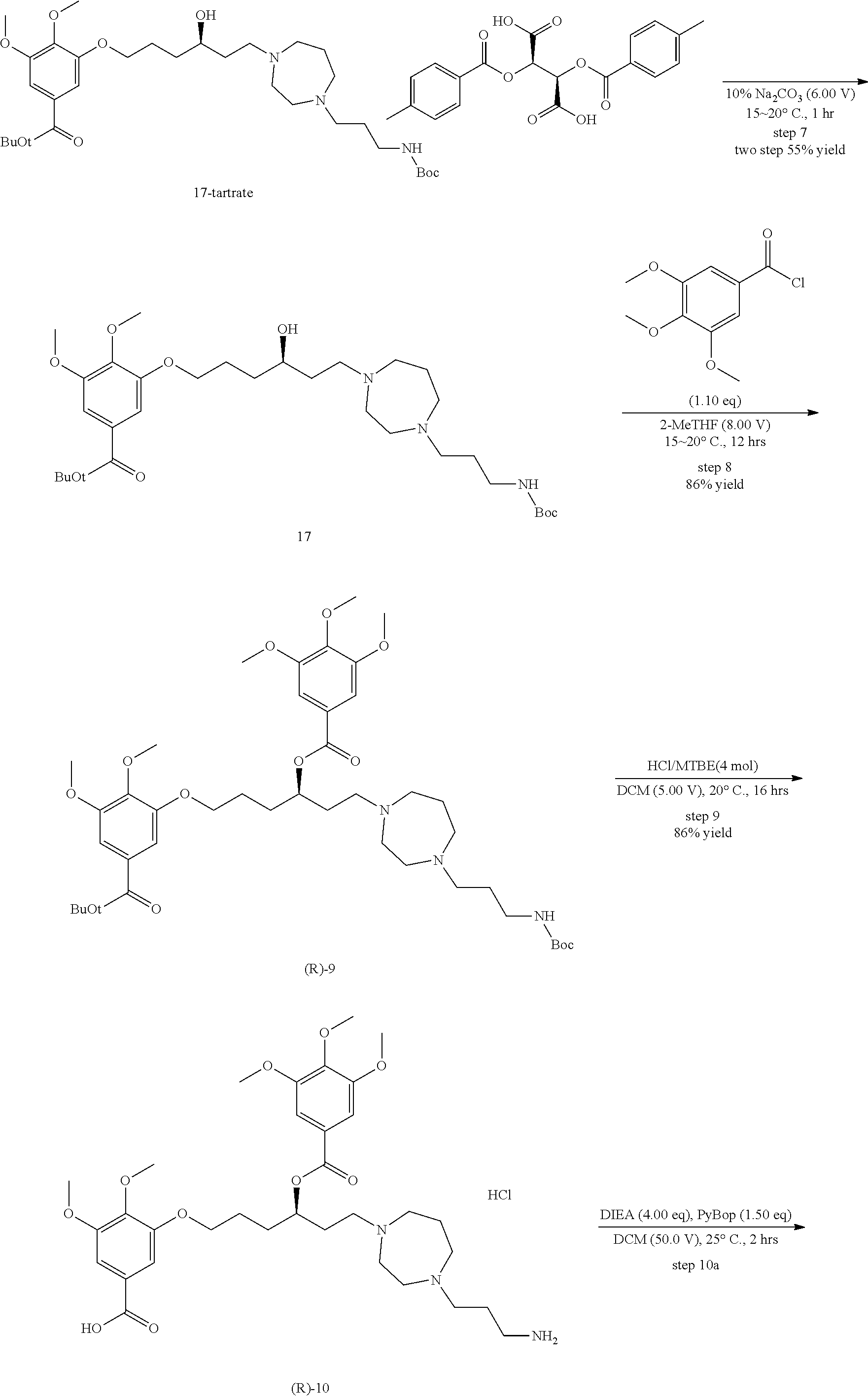

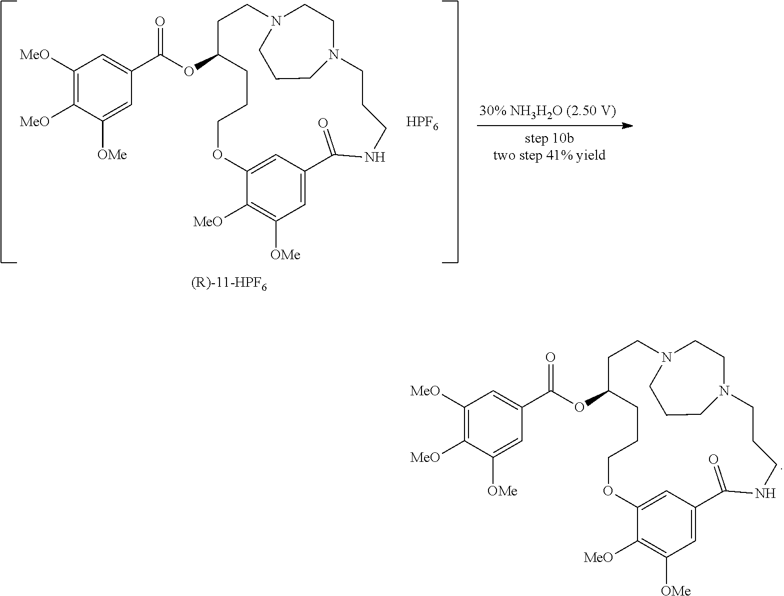

### Step 1:

Compound 12A (1.70 kg, 1.50 eq) was dissolved in THF (5.00 L, 5.00 V). To the reaction mixture was added DIBAL-H (17.4 L, 1 M in toluene, 1.50 eq) dropwise at 0-10° C. The reaction mixture was stirred at 20-30° C. for 2 hrs. The compound 12B (1.00 kg, 1.00 eq) was added to the reaction mixture at 0-10° C. dropwise. The reaction mixture was stirred at 20-30° C. for 12 hrs. H₂O (700 mL, 0.04×mL) was added at 0-10° C. dropwise, then an aqueous solution of NaOH (700 mL, 0.04×mL, 15%) slowly, then additional H₂O (1.74 L, 0.1×mL) at 0-10° C. slowly. The reaction mixture was stirred at 20-30° C. for 15 mins then dried over MgSO₄ (500 g). The solvent was removed under reduced pressure to give the compound 12 (7.80 kg, 65% yield) as yellow oil.

1H NMR: (400 MHZ, CDCl3) δ ppm 1.75-1.95 (m, 2H), 2.53-2.63 (m, 2H), 3.13-3.24 (m, 3H), 3.61-3.72 (m, 5H)

### Step 2:

At 15-25° C., compound 12 (7.80 kg, 1.00 eq), compound 5A (8.30 kg, 1.00 eq), PPh₃ (9.00 kg, 1.05 eq) and toluene (35 L) were charged into the reactor. DEAD (13.0 kg, 1.05 eq) was added slowly to the reaction mixture at 0-10° C. The reaction mixture was stirred at 15-25° C. for 12 hrs. The reaction system was filtered, and the filter cake was washed with MTBE. Water (0.3 L) followed by MgCl₂ (5.64 kg) was added to the filtrate, and the mixture was stirred 20-30° C. for 2 hrs. The reaction system was filtered, and the filter cake was washed with MTBE. The filtrate was washed with 10% citric acid aqueous solution (25.0 L, 3.00× by volume). The organic phase was washed with 5% brine and dried over Na₂SO₄ (4.15 kg, 0.50× by weight). The organic phase was concentrated at 45-55° C. to a volume of 12-20 L. n-Heptane (12.5 L, 1.50× by volume) was added, and the system was reduced to 12-20 L; this was repeated. n-Heptane (41.5 L, 5.00× by volume) was added, and the system was heated at 50-60° C. for 2 hrs with stirring. The system was cooled, filtered and the cake was washed with n-heptane. The filter caked was vacuum dried at 40-50° C., resulting in compound 13 (6.50 kg, 98% purity by HPLC. 65% yield).

1H NMR (400 MHZ, CDCl3-d) δ ppm 7.22-7.26 (m, 2H), 4.08-4.15 (m, 2H), 3.86-3.92 (m, 6H), 3.67-3.71 (m, 3H), 3.16-3.24 (m, 3H), 2.63-2.73 (m, 2H), 2.13-2.25 (m, 2H), 1.57-1.60 (m, 9H)

### Step 3:

At 15-25° C., compound 13 (6.00 kg, 1.00 eq) was dissolved in THF (30.0 L, 5.00 V). At −20° C., compound 3A (39.0 L, 1 M in THF, 6.52× by volume) was added to the reaction mixture slowly. The reaction mixture was stirred at −10-0° C. for 2 hrs. HCl (30.0 L, 1M, 5.00× by volume) was slowly added controlling the pH at 1˜3 at 0˜20° C. MTBE (18.0 L, 3.00× by volume) was added. The organic phase was separated and washed with 0.5 N HCl (18.0 L, 3.00× by volume) twice. The organic phase was washed with 5% NaHCO₃ aqueous (18.0 L, 3.00× by volume), 5% brine (18.0 L, 3.00× by volume), dried over Na₂SO₄, filtered, and concentrated under reduced pressure to give compound 14 (4.50 kg, 75% yield, 92.3% purity) as yellow oil.

1H NMR (400 MHZ, CDCl3-d) δ ppm 7.20-7.26 (m, 2H), 6.20-6.51 (m, 2H), 5.78-5.93 (m, 1H), 4.03-4.17 (m, 2H), 2.80-2.91 (m, 2H), 2.11-2.23 (m, 2H), 1.53-1.64 (m, 9H)

### Step 4:

At 15-25° C., compound 14 (5.20 kg, 1.00 eq) was dissolved in DCM (26.0 L, 5.00× by volume). The compound 8A (4.57 kg, 1.00 eq) and Et₃N (3.0 kg, 2.00 eq) was added and the reaction mixture was stirred for 12 hrs. The reaction mixture was concentrated to give the crude compound 15 (7.30 kg, 90.7% purity 85% yield) as a yellow oil.

1H NMR (400 MHZ, CDCl3) δ ppm 7.17-7.24 (m, 2H), 5.63-5.79 (m, 1H), 4.00-4.07 (m, 2H), 3.81-3.89 (m, 6H), 3.11-3.21 (m, 2H), 2.76-2.84 (m, 2H), 2.44-2.70 (m, 15H), 2.04-2.14 (m, 2H), 1.71-1.79 (m, 2H), 1.57 (s, 9H), 1.41 (s, 9H)

### Step 5:

At 15-25° C., to a solution of compound 15 (5.00 kg, 1.00 eq), HCOOH (7.50 L, 1.50× by volume), Et₃N (7.50 L, 1.50× by volume) in THF (25.0 L, 5.00× by volume) was added (S,S)-Ms-DENEB catalyst (360 g, 0.04 eq). The reaction mixture was stirred at 10-15° C. for 12 hrs. The reaction was cooled to 5-10° C., and pH of the system was adjusted to 11˜12 with saturated Na₂CO₃ aqueous solution (almost 15.0 L). DCM (15.0 L, 3.00 V) was added to the reaction mixture. The organic phase was separated, washed with brine, dried over Na₂SO₄ and concentrated under reduced pressure to give compound 16 (4.50 kg, 83.4% purity, 84.9% ee, 85% yield) as brown oil.

| Instrument: | Shimadzu 20AD | | | | | |
|---|---|---|---|---|---|---|
| Column: | Gemini-NX C18 4.6*150 mm, 5 um | | | | | |
| Column temperature: | 40° C. | | | | | |
| Mobile phase A(MPA) | H₂O + 0.04%(v/v) TFA | | | | | |
| Mobile phase B(MPB) | ACN + 0.02%(v/v) TFA | | | | | |
| Flow rate: | 1.2 mL/min | | | | | |
| | Time(min) | 0.01 | 16 | 19 | 19.01 | 20.00 |
| Gradient | MPA(%) | 90 | 20 | 0 | 90 | 90 |
| Ratio: | MPB(%) | 10 | 80 | 100 | 10 | 10 |

| | |
|---|---|
| Detection: | 220 nm 215 nm 254 nm |

### Steps 6-7:

The compound 16 (62.0 kg, 1.00× by weight) was dissolved in acetone (ACE) (434 L, 7.00× by volume) and EtOH (434 L, 7.00× by volume) at 10-20° C. then the mixture was stirred at 50-55° C. for 1 hr and then allowed to cool to 25-30° C. acetone (186 L, 3.00× by volume), ethanol (186 L, 3.00× by volume) and cpd. 6A (78.5 kg, 1.26× by weight) were added and the mixture stirred at 50˜55° C. for 1 hr. The system was cooled to 25˜30° C. at a rate of 3˜5° C. degrees an hour. The mixture was filtered, and the filter cake was washed with ACE:EtOH=1:1 (4.50 L, 1.00× by volume) and dried under N₂ in a blast drying oven at 45˜55° C. affording the product 17-tartrate (5.10 kg, 98.0% purity, 97.5% ee). The salt compound 17-tartrate is converted to the corresponding acid (compound 17).

### Step 7:

Compound 17 (1.50 kg, 1.0 equiv.) was dissolved in 2-MeTHF (20.2 L). Under N₂, the solution was cooled to 10-15° C. A solution of 2, 3, 4 trimethoxybenzoyl chloride (624.0 g, 1.10 equiv) in 2-MeTHF (3.00 L) was added to the solution dropwise. The reaction was stirred at 15-20° C. for 16 h. At which time, aqueous Na₂CO₃ (10%, 4.5 L) was added to adjust the pH to 11-12 at 10-20° C. The organic phase was separated, washed with 10% NaCl (4.5 L), dried over Na₂SO₄, and filtered. The solvent of the filtrate was removed under reduced pressure to give compound (R)-9 (1.70 kg, 94.4% purity, 97.7% ee, 86% yield).

### Step 8:

Compound (R)-9 (200 g, 1.00 equiv) was dissolved in DCM (1.00 L) under N₂. At 15-20° C., 4M HCl in MTBE (600 mL) was added. The reaction mixture was stirred at 15-20° C. for 16 hrs. MTBE (2.00 L) was added dropwise over 20 min. A white precipitate formed. Stirring was ceased, and the mixture was allowed to stand for 30 min. The supernatant liquid was removed with a peristalic pump, reducing the solution to a volume of ˜1.0 L. The mixture was filtered, and the filter cake was dried under vacuum at 40-45° C. to afford compound (R)-10 (150 g, 98.6 purity, 86% yield).

### Steps 9-10:

PyBOP (913 g, 1.5 eq) was dissolved in DCM (40.0 L, 30.0 V) under N₂ at 15˜25° C. DIEA (600 g, 4.00 eq) was added followed by a solution of compound (R)-10 (800 g, 1.00 eq) in DCM (1.60 L, 20.0 V) over about 1.5 hrs. The mixture was stirred for an additional 20 min at 15˜25° C. At which time, the reaction was concentrated to ˜2.00 V at 40-45°. The solution was washed with water (2.40 L, 3.00 V) three times. The organic phase was washed with 10% NaCl (2.40 L, 3.00 V). The organic phase was dried over Na₂SO₄ (200 g, 0.25× by weight), and filtered. The filter cake was washed with MeOH. MeOH (2.40 L, 3.00 V) was added into the filtrate and then the mixture was concentrated to about 2.00 V. The addition of MeOH and concentration was repeated two more times to remove any residual DCM. MeOH (2.40 L, 3.00 V) was added into mixture and stirred at 15˜25° C. for 12 hrs. The system was filtered and the resultant cake was washed with MeOH (0.80 L, 1.00 V). The filter cake was dried cake under vacuum at 45˜50° C. and 550 g of compound (R)-11-HPF₆ was obtained with 98.8% purity (66% yield).

A reaction vessel was charged with MeOH (4.00 L, 5.00 V) and 550 g of compound (R)-11 HPF₆. The reaction vessel was subsequently charged with 30% of NH₃·H₂O (1375 mL, 2.50 V) slowly at 15˜25° C. for 20 mins until the system gradually became clear. The system was extracted with DCM three times (2750 mL, (5.00 V)×3). The organic layers were combined, washed with 10% of Na₂CO₃ (1.50 L, 3.00 V) one time, and washed with 10% NaCl (1.50 L, 3.00 V). The organic layer was dried over Na₂SO₄ (125 g, 0.25× by weight), filtered (washing the filter cake with DCM (250 mL, 0.50 V)) and solvent was removed under vacuum to give 410 g of crude compound (R)-11.

### Example 4. Synthesis of Compound (R)-11

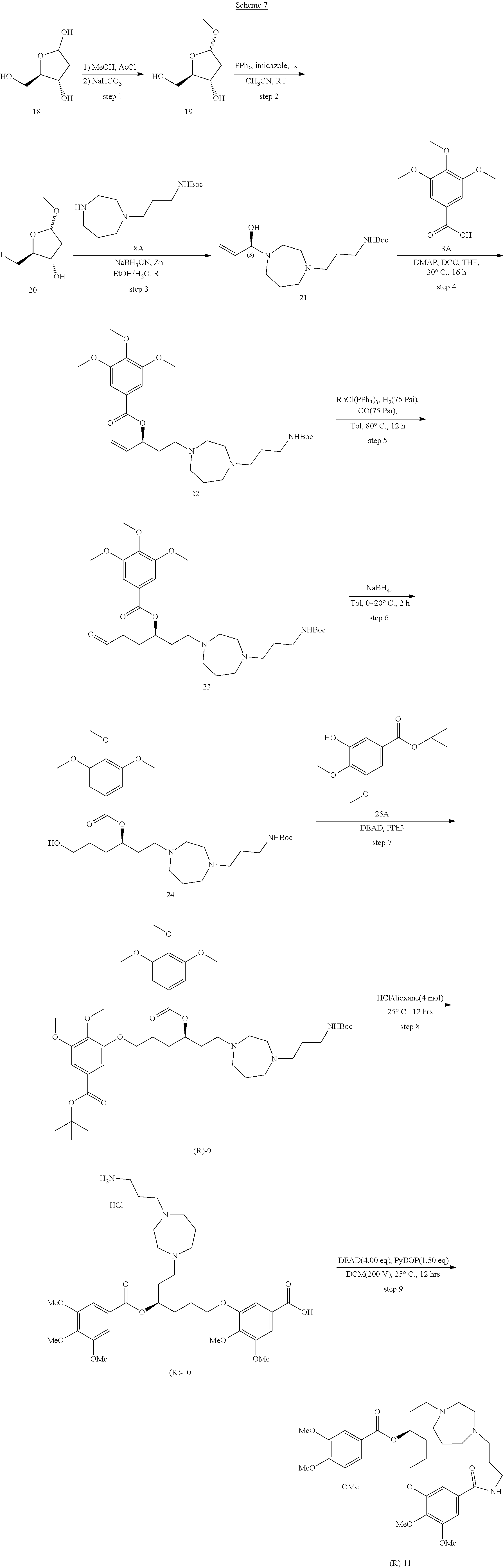

### Step 1:

Methanol (1.2 L) is charged into a reactor, stirred for 10-15 minutes, and cooled to 0-5° C., then acetyl chloride (2.34 g, 29.8 mmol, 0.05 eq) is added and the mixture is stirred for 10-15 minutes at 0-5° C. The obtained methanolic hydrogen chloride is transferred into another container. Methanol (400 mL) is charged into a clean reactor and stirred for 10-15 minutes at 25-35° C. 2-deoxy-D-ribose (80.0 g, 596.43 mmol, 1.00 eq) is charged into the reactor and the mixture is stirred at 25-35° C. for 10-15 minutes. The mass is cooled to 0-5° C. and the methanolic hydrogen chloride solution prepared above is charged into the reactor at same temperature. The obtained mass is maintained at 0-5° C. for 2-3 hours. Sodium bicarbonate (3.0 g, 35.78 mmol, 0.06 eq) is charged into the mass at 0-5° C. and the mass is filtered. The filtrate is collected in another container and the filter bed is washed with methanol (100 mL). The combined filtrate was concentrated. The residue was purified by silica gel column chromatography, eluted with PE/THF (5:1) to afford (2R,3S)-2-(hydroxymethyl)-5-methoxyoxolan-3-ol (19) (83 g, 94% yield) as a white solid.

### Step 2:

To a stirred solution of (2R,3S)-2-(hydroxymethyl)-5-methoxyoxolan-3-ol (80 g, 539.96 mmol, 1.00 equiv) and PPh₃ (212.44 g, 0.81 mol, 1.50 equiv) in THF (1.6 L) were added imidazole (73.52 g, 1.08 mol, 2.00 equiv) and I₂ (205.57 g, 0.81 mol, 1.50 equiv) at room temperature under nitrogen atmosphere. The resulting mixture was stirred for 16 h at room temperature under nitrogen atmosphere. The reaction was quenched with a saturated solution of NaHSO₃ at room temperature. The organic phase was washed with brine. The organic layers were dried over anhydrous Na₂SO₄ and concentrated under reduced pressure. The residue was purified by silica gel column chromatography, eluted with PE/THF (5:1) to afford (2S,3S)-2-(iodomethyl)-5-methoxyoxolan-3-ol (98 g, 70% yield) as light oil.

### Step 3:

To a stirred solution of (2S,3S)-2-(iodomethyl)-5-methoxyoxolan-3-ol (6.1 g, 23.63 mmol, 1.00 equiv) and zinc (15.46 g, 236.38 mmol, 10.00 equiv) in EtOH (120 mL) and AcOH (1.7 g, 28.36 mmol, 1.20 equiv) were added tert-butyl(S)-(3-(4-(3-hydroxypent-4-en-1-yl)-1,4-diazepan-1-yl) propyl) carbamate (4.73 g, 23.63 mmol, 1.00 equiv) and NaBH₃CN (4.46 g, 70.91 mmol, 3.00 equiv) dropwise at room temperature. The resulting mixture was stirred for 2 h at room temperature. The resulting mixture was diluted with DCM (20 mL). The resulting mixture was filtered; the filter cake was washed with DCM (10 mL). The filtrate was concentrated under reduced pressure. The residue was purified by silica gel column chromatography, eluted with PE:THF (1:2) to afford tert-butyl 4-[(3S)-3-hydroxypent-4-en-1-yl]-1,4-diazepane-1-carboxylate (2.8 g, 42% yield) as a colorless oil.

LC-MS (ES+) m/z: 285.2 (M+H)+ (calculated: 285.2)

### Step 4:

To a solution of tert-butyl(S)-(3-(4-(3-hydroxypent-4-en-1-yl)-1,4-diazepan-1-yl) propyl) carbamate (1, 996.47 mg, 3.50 mmol, 1 eq) and 3,4,5-trimethoxybenzoic acid (3A) (2, 891.24 mg, 4.20 mmol, 1.2 eq) in THF (10 mL) was added DCC (1.08 g, 5.25 mmol, 1.06 mL, 1.5 eq) and DMAP (641.38 mg, 5.25 mmol, 1.5 eq). The mixture was stirred at 30° C. for 16 hr. The residue was purified by column chromatography (SiO₂, Petroleum ether/Ethyl acetate=100/1 to 1/1), TLC (Petroleum ether/Ethyl acetate=2:1, Rf-0.42) to afford(S)-5-(4-(3-((tert-butoxycarbonyl)amino) propyl)-1,4-diazepan-1-yl) pent-1-en-3-yl 3,4,5-trimethoxybenzoate (3, 1.3 g, 2.53 mmol, 72.18% yield) as a white solid.

LC-MS (ES+) m/z: 479.0 (M+H)+ (calculated: 479.3)

1H NMR (400 MHZ, CDCl3-d) δ 7.31 (s, 2H), 5.91 (ddd, J=6.4, 10.4, 17.1 Hz, 1H), 5.56 (q, J=6.5 Hz, 1H), 5.34 (br d, J=17.4 Hz, 1H), 5.23 (br d, J=10.5 Hz, 1H), 4.81 (br d, J=2.0 Hz, 6H), 4.15-4.04 (m, 2H), 3.91 (d, J=1.2 Hz, 12H), 3.51-3.37 (m, 6H), 2.70-2.53 (m, 8H), 1.45 (s, 12H)

### Step 5:

To a solution of(S)-5-(4-(3-((tert-butoxycarbonyl)amino) propyl)-1,4-diazepan-1-yl) pent-1-en-3-yl 3,4,5-trimethoxybenzoate (3, 100 mg, 208.95 μmol, 1 eq) in toluene (5 mL) was added chlororhodium; triphenylphosphane (19.33 mg, 20.90 μmol, 0.1 eq) under N₂ atmosphere. The mixture was stirred under H₂ (75 Psi) and CO (75 Psi) at 80° C. for 12 hr. The reaction mixture was filtered and concentrated under reduced pressure to afford (R)-1-(4-(3-((tert-butoxycarbonyl)amino) propyl)-1,4-diazepan-1-yl)-6-oxohexan-3-yl 3,4,5-trimethoxybenzoate (100 mg, crude) as a brown oil. The residue was used to in the next step without purification. LC-MS (ES+) m/z: 509.2 (M+H)+ (calculated: 509.3)

### Step 6:

To a solution of (R)-1-(4-(3-((tert-butoxycarbonyl)amino) propyl)-1,4-diazepan-1-yl)-6-oxohexan-3-yl 3,4,5-trimethoxybenzoate (4, 100 mg, 247.19 μmol, 1 eq) in toluene (2 mL) was added NaBH₄ (14.03 mg, 370.79 μmol, 1.5 eq) at 0° C. The mixture was stirred at 20° C. for 1 hr. The residue was purified by prep-HPLC (column: Phenomenex Luna C18 150*25 mm*10 μm; mobile phase: [water (FA)-ACN]; B %: 12%-42%, 10 min) to afford (R)-1-(4-(3-((tert-butoxycarbonyl)amino) propyl)-1,4-diazepan-1-yl)-6-hydroxyhexan-3-yl 3,4,5-trimethoxybenzoate (10 mg, 9.26% yield) as a white solid.

LC-MS (ES+) m/z: 511.1 (M+H)+ (calculated: 511.3)

1H NMR (400 MHZ, CDCl3-d) δ 8.40 (br s, 1H), 7.28 (s, 2H), 5.22 (quin, J=6.1 Hz, 1H), 3.94-3.91 (m, 9H), 3.72-3.65 (m, 3H), 3.62 (br s, 1H), 3.53-3.44 (m, 2H), 2.97 (br s, 3H), 2.88 (br d, J=7.4 Hz, 3H), 2.13 (br s, 4H), 1.91-1.77 (m, 2H), 1.73-1.59 (m, 2H), 1.46 (s, 9H)

### Step 7:

At 20° C., compound 24 (1.00 eq) and Tol, compound 25A (0.85 kg, 3.34 mol, 1.00 eq), and PPh₃ (1.05 eq) were charged into the reaction. DEAD (1.00 eq) was added dropwise, (Exothermic phenomenon is observed during the addition process). After addition, the reaction mixture was stirred at 25° C. for 6 hrs, then the reaction mixture was stirred at −20° C. for 1 hr to precipitate part of OPPh₃. The reaction mixture was filtered and the filtrate concentrated under reduced pressure to give crude product. The crude product was purified by silica gel chromatography.

1H NMR (400 MHZ, CDCl3-d) δ 7.30 (s, 2H), 7.21 (s, 2H), 5.23-5.36 (m, 1H), 4.04-4.17 (m, 2H), 3.73-3.94 (m, 15H), 3.06 (t, J=6.8 Hz, 2H), 2.65-2.80 (m, 8H), 2.60 (t, J=7.6 Hz, 2H), 2.49 (t, J=7.6 Hz, 2H), 1.86-2.03 (m, 6H), 1.76-1.85 (m, 2H), 1.61-1.68 (m, 2H), 1.58 (s, 9H), 1.43 (s, 9H).

### Step 8:

At 0-5° C., a solution of HCl in dioxane (4 mol, 7.60 L) and compound (R)-9 (1086 g, 1.35 mol, 1.00 eq) was charged into a 20.0 L reactor. The reaction mixture was stirred at 25° C. for 12 hrs. The solvent was removed under reduced pressure to give the compound (R)-10 (1050 g, as HCl Salt) as yellow solid.

Purity determined by quantitative NMR: 75.2%

1H NMR (400 MHZ, MeOD-d4) δ 7.29 (s, 2H), 7.26 (s, 2H) 5.2-5.37 (m, 1H), 4.11 (s, 2H), 3.94 (brs, 4H), 3.78-3.90 (m, 15H), 3.33-3.45 (m, 4H), 3.08 (t, J=7.6 Hz, 2H), 2.12-2.49 (m, 6H), 1.90-2.09 (m, 4H)

### Step 9:

At 20° C., compound (R)-10 (1050 g, 1.53 mol, 1.00 eq, HCl) and DCM (210 L) were charged into the reactor. Then, DIEA (793 g, 6.13 mol, 4.00 eq) and PyBOP (38.4 g, 2.29 mol, 1.50 eq) were added to the reactor at 20° C. The reaction mixture was stirred at 25° C. for 12 hrs. The reaction mixture was concentrated at 35-40° C. to give the residue. The residue was triturated with MeOH (4.2 L, 4.00 V) at 20° C. for 60 min. The mixture was filtered and the cake collected to give the compound (R)-11 470 g, 34.94 mmol, 48.6% yield) as white solid. Purity determined by quantitative NMR: 75.2%

1H NMR (400 MHZ, MeOD-d4) δ 7.31 (s, 2H), 7.20 (d, J=1.8 Hz, 1H), 7.13 (d, J=1.8 Hz, 1H), 5.49 (s, 1H), 4.31 (br d, J=8.3 Hz, 1H), 4.18 (br s, 1H), 3.85-3.89 (m, 9H), 3.81 (d, J=7.3 Hz, 6H), 3.56-3.66 (m, 1H), 3.38-3.49 (m, 1H), 2.97 (td, J=3.2, 10.3 Hz, 1H), 2.84-2.91 (m, 2H), 2.74-2.84 (m, 3H), 2.61-2.73 (m, 4H), 2.56 (br t, J=6.5 Hz, 2H), 1.86-1.95 (m, 5H), 1.73-1.85 (m, 5H).

1. A process for preparing compound (R)-11, or a pharmaceutically acceptable salt or solvate thereof, 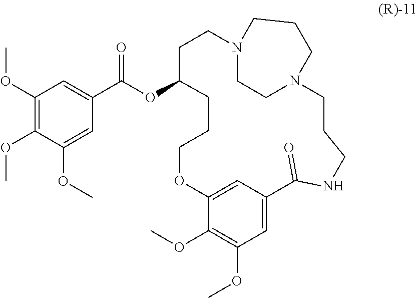 [SMILES: COc1cc(C(=O)O[C@@H]2CCCOc3cc(cc(OC)c3OC)C(=O)NCCCN3CCCN(CC2)CC3)cc(OC)c1OC]
comprising the step of separating compound (R)-11 from a racemic mixture of compound 11:

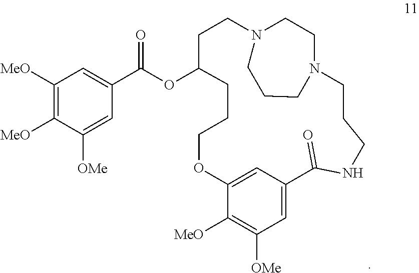 [SMILES: COc1cc(C(=O)OC2CCCOc3cc(cc(OC)c3OC)C(=O)NCCCN3CCCN(CC2)CC3)cc(OC)c1OC]

2. (canceled)

3. The process of claim 1, further comprising the step of reacting a salt of compound 10 with a peptide coupling reagent to prepare compound 11:

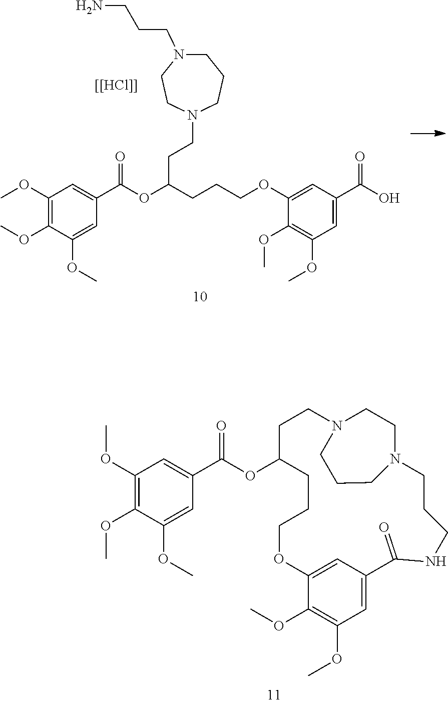

4. (canceled)

5. (canceled)

6. The process of claim 3, further comprising the step of deprotecting compound 9 to prepare a salt of compound 10 by reacting compound 9 with an acid:

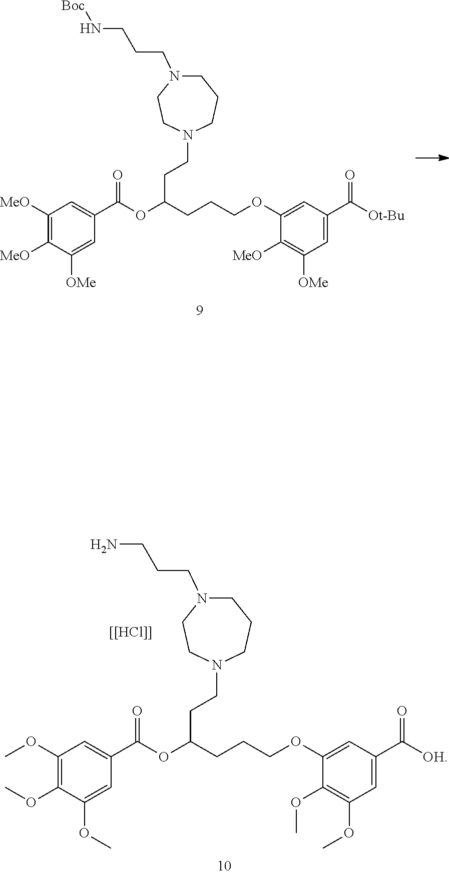

7. (canceled)

8. The process of claim 6, further comprising the step of reacting compound 8 with compound 8A to prepare compound 9:

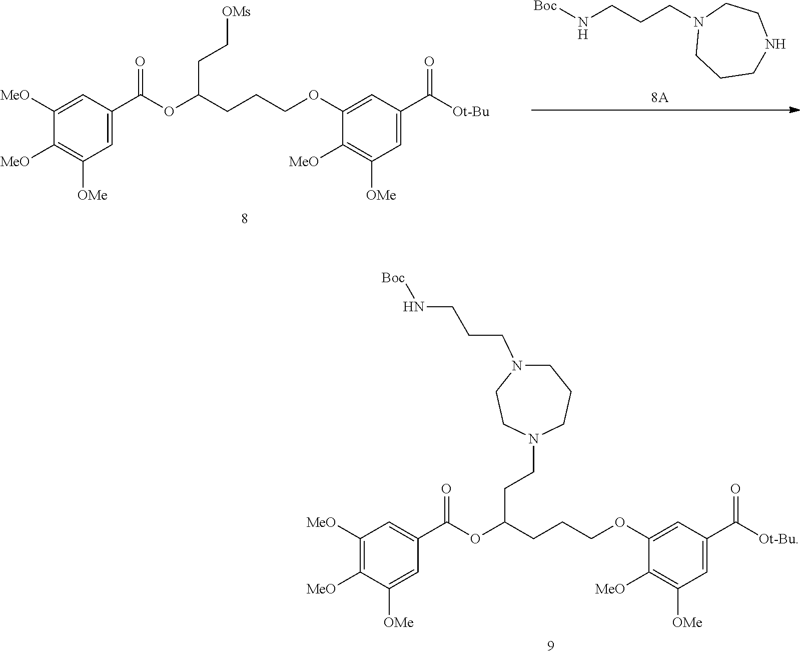 [SMILES: COc1cc(C(=O)OC(CCCOc2cc(C(=O)OC(C)(C)C)cc(OC)c2OC)CCOS(C)(=O)=O)cc(OC)c1OC ; CC(C)(C)OC(=O)NCCCN1CCCNCC1 ; COc1cc(C(=O)OC(CCCOc2cc(C(=O)OC(C)(C)C)cc(OC)c2OC)CCN2CCCN(CCCNC(=O)OC(C)(C)C)CC2)cc(OC)c1OC]

9. (canceled)

10. (canceled)

11. (canceled)

12. The process of claim 8, further comprising the step of reacting compound 7 with a mesylating agent to prepare compound 8:

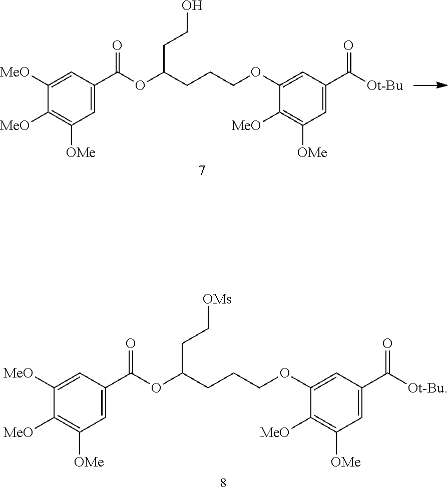 [SMILES: COc1cc(C(=O)OC(CCO)CCCOc2cc(C(=O)OC(C)(C)C)cc(OC)c2OC)cc(OC)c1OC ; COc1cc(C(=O)OC(CCCOc2cc(C(=O)OC(C)(C)C)cc(OC)c2OC)CCOS(C)(=O)=O)cc(OC)c1OC]

13. (canceled)

14. (canceled)

15. (canceled)

16. (canceled)

17. The process of claim 12, further comprising reacting compound 6 with a suitable deprotecting agent to prepare compound 7:

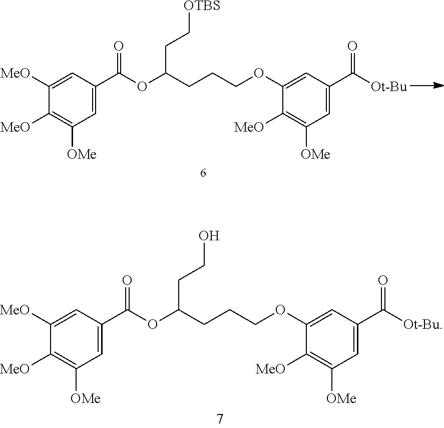 [SMILES: COc1cc(C(=O)OC(CCCOc2cc(C(=O)OC(C)(C)C)cc(OC)c2OC)CCO[Si](C)(C)C(C)(C)C)cc(OC)c1OC ; COc1cc(C(=O)OC(CCO)CCCOc2cc(C(=O)OC(C)(C)C)cc(OC)c2OC)cc(OC)c1OC]

18. (canceled)

19. (canceled)

20. (canceled)

21. The process of claim 17, further comprising the step of reacting compound 5 with compound 5A to prepare compound 6

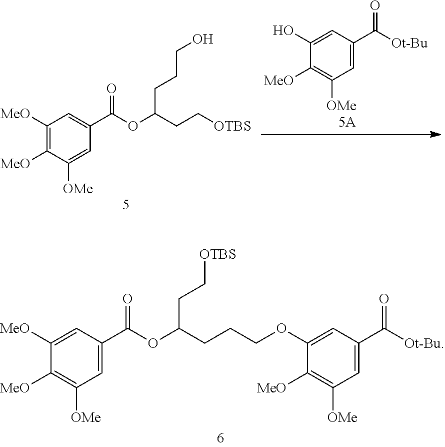 [SMILES: COc1cc(C(=O)OC(CCCO)CCO[Si](C)(C)C(C)(C)C)cc(OC)c1OC ; COc1cc(C(=O)OC(C)(C)C)cc(O)c1OC ; COc1cc(C(=O)OC(CCCOc2cc(C(=O)OC(C)(C)C)cc(OC)c2OC)CCO[Si](C)(C)C(C)(C)C)cc(OC)c1OC]

22. (canceled)

23. The process of claim 21, further comprising the step of preparing compound 5 from compound 4:

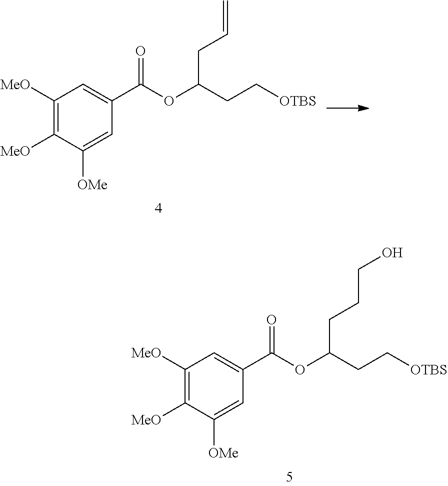 [SMILES: C=CCC(CCO[Si](C)(C)C(C)(C)C)OC(=O)c1cc(OC)c(OC)c(OC)c1 ; COc1cc(C(=O)OC(CCCO)CCO[Si](C)(C)C(C)(C)C)cc(OC)c1OC]

24. (canceled)

25. (canceled)

26. The process of claim 23, further comprising the step of reacting compound 3 with compound 3A to prepare compound 4:

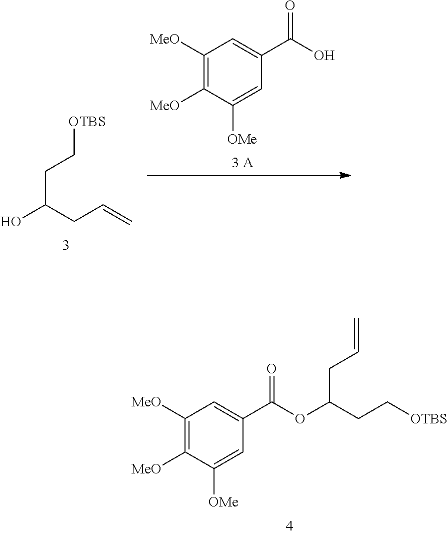 [SMILES: C=CCC(O)CCO[Si](C)(C)C(C)(C)C ; COc1cc(C(=O)O)cc(OC)c1OC ; C=CCC(CCO[Si](C)(C)C(C)(C)C)OC(=O)c1cc(OC)c(OC)c(OC)c1]

27. (canceled)

28. (canceled)

29. (canceled)

30. (canceled)

31. (canceled)

32. (canceled)

33. A process of preparing a compound (R)-11, or a pharmaceutically acceptable salt or solvate thereof, comprising the step of reacting compound 18 with compound 3A:

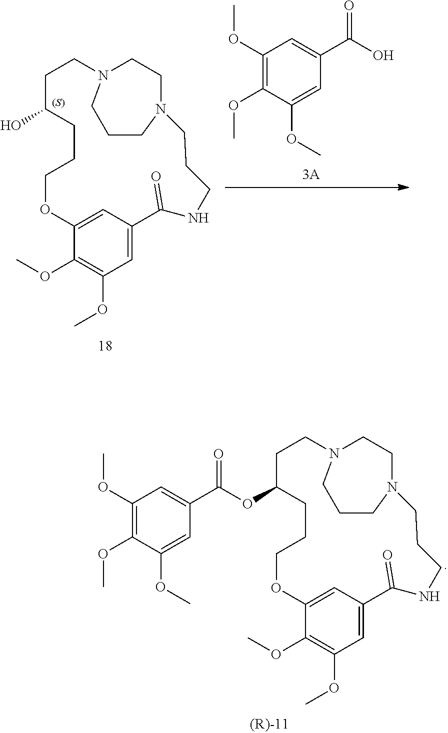

34. (canceled)

35. (canceled)

36. (canceled)

37. (canceled)

38. A process for preparing compound (R)-11, or a pharmaceutically acceptable salt or solvate thereof, 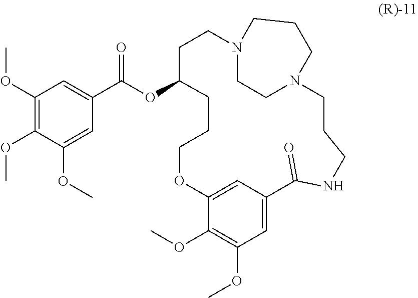 [SMILES: COc1cc(C(=O)O[C@@H]2CCCOc3cc(cc(OC)c3OC)C(=O)NCCCN3CCCN(CC2)CC3)cc(OC)c1OC]
comprising the step of reacting a salt of compound (R)-10 with a peptide coupling reagent:

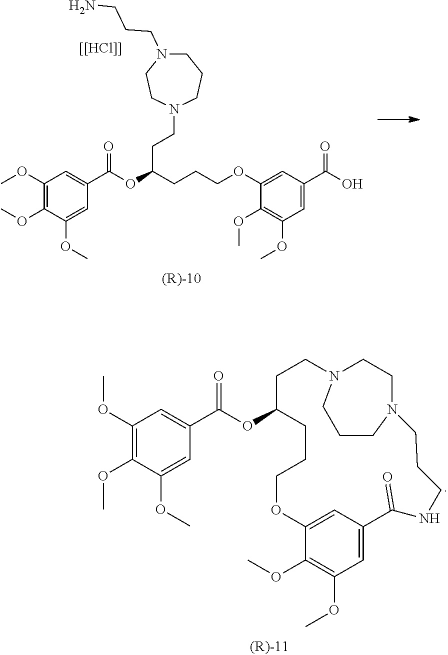

39. (canceled)

40. (canceled)

41. The process of claim 38, further comprising the step of deprotecting compound 9 to prepare a salt of compound (R)-10 by reacting compound (R)-9 with an acid:

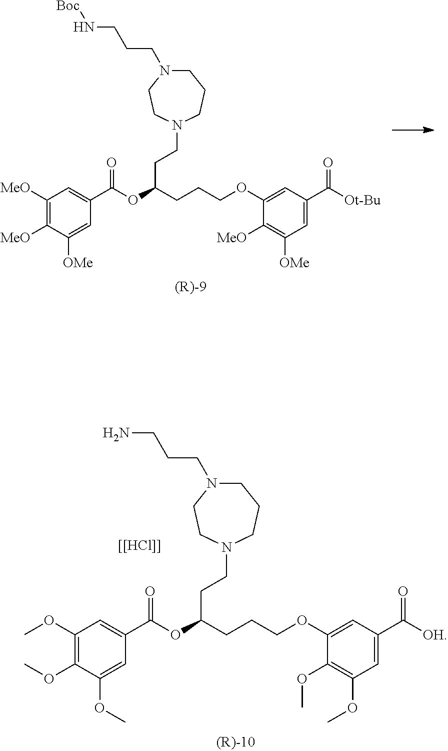

42. (canceled)

43. A process of preparing compound (R)-9, further comprising the step of reacting compound (R)-17 with 3,4,5-trimethoxybenzoic acid or 3,4,5-trimethoxybenzoyl chloride:

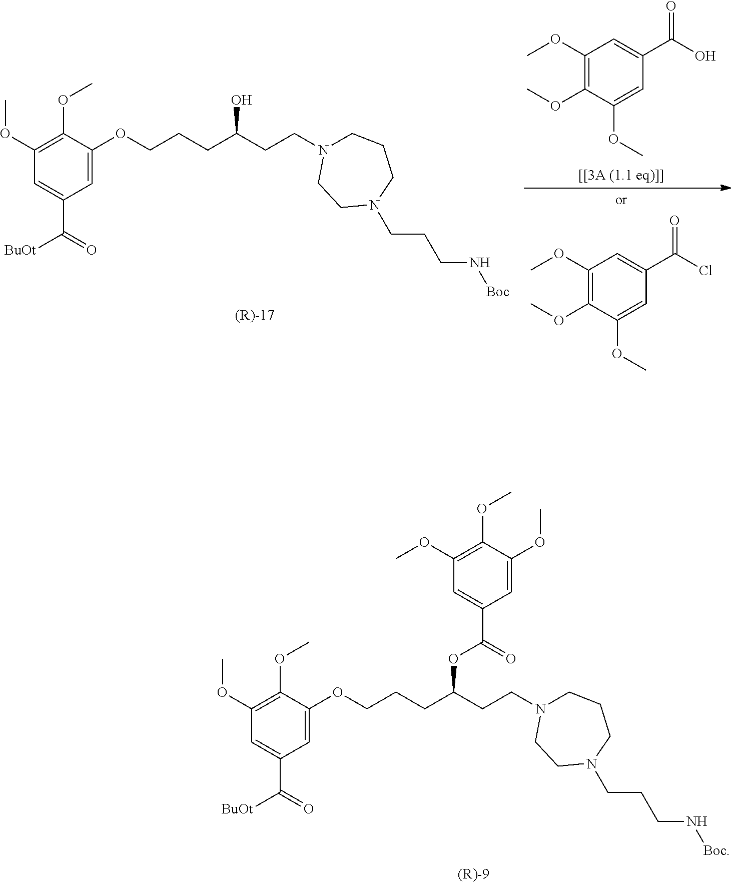

44. (canceled)

45. (canceled)

46. (canceled)

47. (canceled)

48. (canceled)

49. The process of claim 43, further comprising reacting compound 15 with a suitable reducing agent to prepare compound (R)-17:

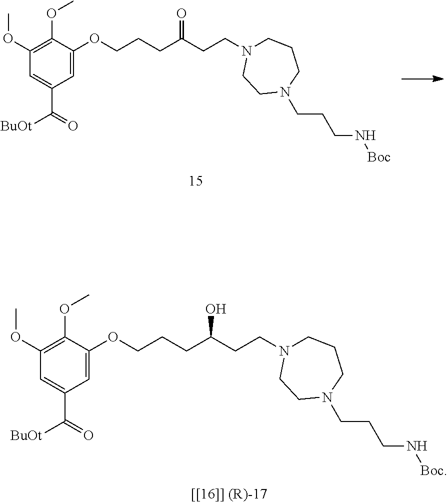

50. (canceled)

51. (canceled)

52. The process of claim 49, further comprising the step of reacting compound 14 with compound 8A to prepare compound 15:

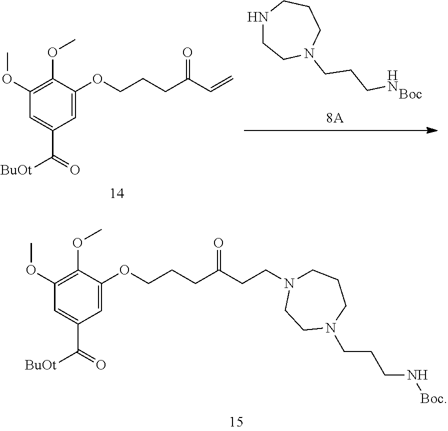

53. (canceled)

54. (canceled)

55. The process of claim 52, further comprising the step of preparing compound 14 from compound 13:

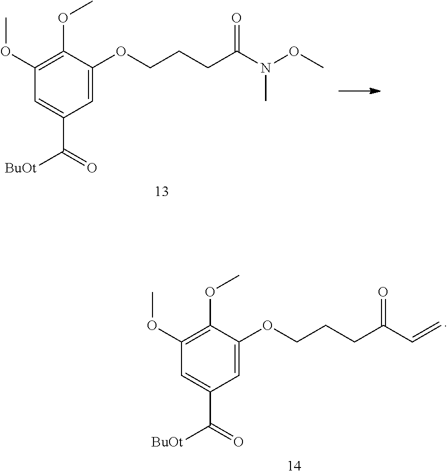

56. The process of claim 55, further comprising the step of reacting compound 12 with compound 5A to prepare compound 13:

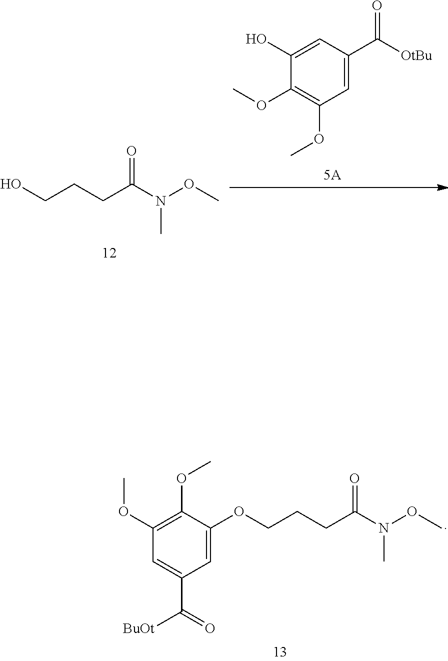

57. (canceled)

58. (canceled)

59. (canceled)

60. (canceled)

61. (canceled)

62. (canceled)

63. (canceled)

64. (canceled)

65. (canceled)

66. (canceled)

67. A process preparing compound (R)-9, comprising the step of reacting compound 24 with compound 25A:

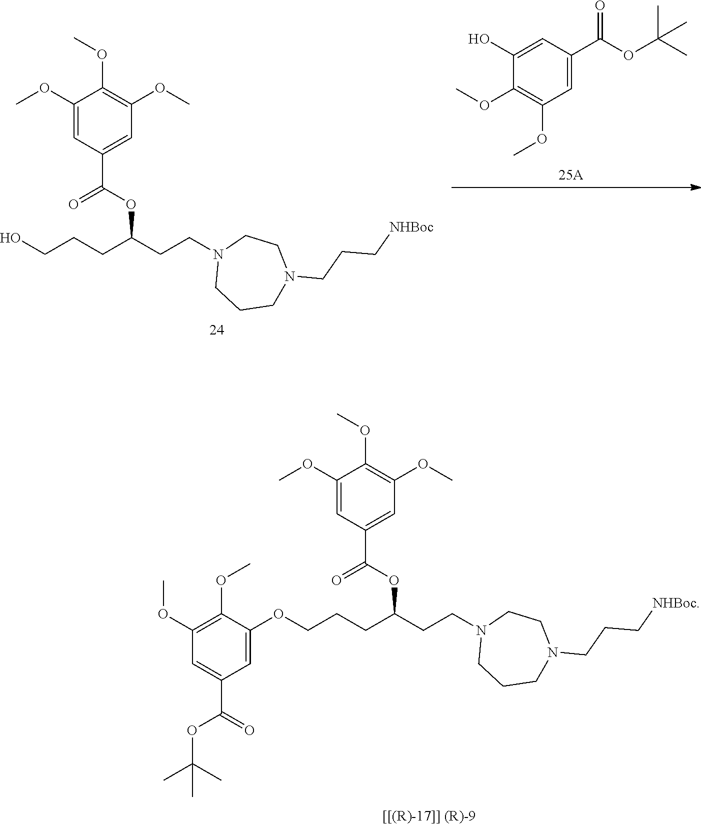 [SMILES: COc1cc(C(=O)O[C@H](CCCO)CCN2CCCN(CCCNC(=O)OC(C)(C)C)CC2)cc(OC)c1OC ; COc1cc(C(=O)OC(C)(C)C)cc(O)c1OC ; COc1cc(C(=O)O[C@H](CCCOc2cc(C(=O)OC(C)(C)C)cc(OC)c2OC)CCN2CCCN(CCCNC(=O)OC(C)(C)C)CC2)cc(OC)c1OC]

68. (canceled)

69. (canceled)

70. (canceled)

71. The process of claim 67, further comprising the step of reacting compound 23 with a reducing agent to prepare compound 24:

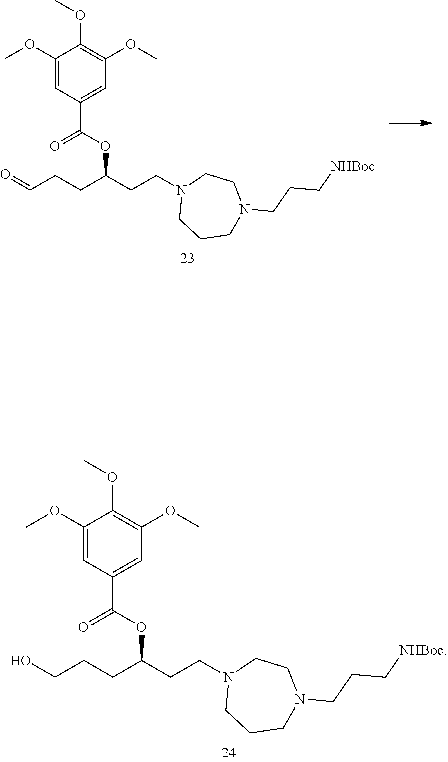 [SMILES: COc1cc(C(=O)O[C@H](CCC=O)CCN2CCCN(CCCNC(=O)OC(C)(C)C)CC2)cc(OC)c1OC ; COc1cc(C(=O)O[C@H](CCCO)CCN2CCCN(CCCNC(=O)OC(C)(C)C)CC2)cc(OC)c1OC]

72. (canceled)

73. The process of claim 71, further comprising the step of reacting compound 22 with a catalyst to prepare compound 23:

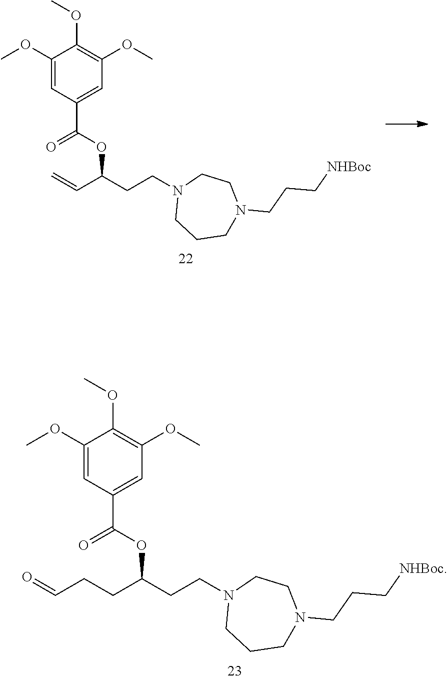 [SMILES: C=C[C@H](CCN1CCCN(CCCNC(=O)OC(C)(C)C)CC1)OC(=O)c1cc(OC)c(OC)c(OC)c1 ; COc1cc(C(=O)O[C@H](CCC=O)CCN2CCCN(CCCNC(=O)OC(C)(C)C)CC2)cc(OC)c1OC]

74. (canceled)

75. The process of claim 73, further comprising the step of reacting compound 21 with compound 3A to prepare compound 22:

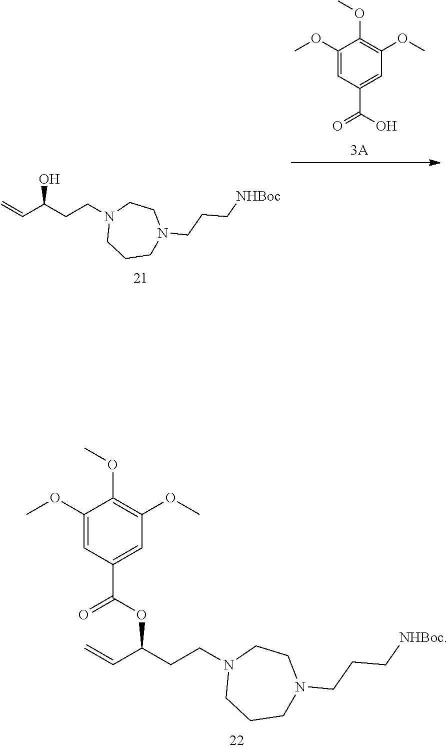

76. (canceled)

77. (canceled)

78. The process of claim 75, further comprising the step of reacting compound 20 with compound 8A to prepare compound 21:

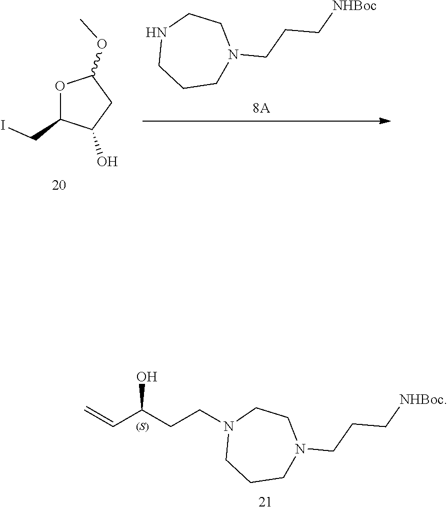

79. (canceled)

80. (canceled)

81. (canceled)

82. (canceled)

83. (canceled)
